(19) United States
(12) Patent Application Publication
(10) Pub. No.: US 2026/0174908 A1
(43) Pub. Date: Jun. 25, 2026

(54) COMPOSITIONS AND METHODS OF TREATING, PREVENTING, OR DELAYING THE PROGRESSION OF NEURODEGENERATIVE DISEASE

(71) Applicant: ARIZONA BOARD OF REGENTS ON BEHALF OF THE UNIVERSITY OF ARIZONA, Tucson, AZ (US)

(72) Inventors: Fei Yin, Tucson, AZ (US); Weihua Wang, Tucson, AZ (US)

(21) Appl. No.: 19/548,223

(22) Filed: Feb. 24, 2026

Related U.S. Application Data
(63) Continuation-in-part of application No. PCT/US24/42331, filed on Aug. 14, 2024.
(60) Provisional application No. 63/578,608, filed on Aug. 24, 2023.

(51) Int. Cl. A61K 48/00; A61K 38/17; A61P 25/28

(52) U.S. Cl. CPC A61K 48/0058; A61K 38/1783; A61P 25/28; C12N 2750/14143; C12N 2750/14145

(57) ABSTRACT

Methods and compositions for manipulating metabolism in astrocytes to improve astrocytic and neuronal function, as well as individual pathology hallmarks and symptoms during the development of neurodegeneration by increasing PPARα expression and activity specifically in astrocytes within the central nervous system to treat a variety of neurodegenerative conditions including but not limited to conditions involving neuronal lipid dysregulation, oxidative damage, accumulation of β-amyloid or other protein aggregates, dementia, and motor dysfunction. Also provided are methods of treating peripheral metabolic dysfunction associated with neurodegenerative conditions.

### CROSS-REFERENCE TO RELATED APPLICATIONS

This application is a continuation-in-part and claims benefit of PCT Application No. PCT/US24/42331 filed Aug. 14, 2024, which claims benefit of U.S. Provisional Application No. 63/578,608 filed Aug. 24, 2023, the specifications of which are incorporated herein in their entirety by reference.

### STATEMENT REGARDING FEDERALLY SPONSORED RESEARCH OR DEVELOPMENT

This invention was made with government support under Grant Nos. AG068175 and AG079157 awarded by National Institutes of Health. The government has certain rights in the invention.

### REFERENCE TO AN ELECTRONIC SEQUENCE LISTING

The contents of the electronic sequence listing (ARIZ_23_14_PCT_CIP.xml; Size: 27,453 byte bytes; and Date of Creation: Feb. 23, 2026) is herein incorporated by reference in its entirety.

### FIELD OF THE INVENTION

The present invention is in the field of molecular biology, more particularly, the present invention relates to methods and compositions for manipulating metabolism in astrocytes to improve astrocytic and neuronal function, as well as individual pathology hallmarks and symptoms during the development of neurodegeneration. In particular, it relates to increasing PPARα expression and activity, specifically in astrocytes within the central nervous system, to treat a variety of neurodegenerative conditions, and specifically those which cause neuronal lipid dysregulation, synaptic loss, oxidative damages, accumulation of β-amyloid or other protein aggregates, dementia, and motor dysfunction.

### BACKGROUND OF THE INVENTION

Neurodegenerative diseases, including but not limited to Alzheimer's disease (AD), Parkinson's disease (PD), Huntington's disease (HD), amyotrophic lateral sclerosis (ALS), multiple sclerosis (MS), and prion diseases, are increasingly being recognized to share common cellular and molecular mechanisms related to abnormal lipid metabolism. Fatty acids are the essential component of most lipid species. The brain critically depends on astrocytes to eliminate fatty acids (FAs) and maintain lipid balance. Recently, it was revealed that impaired FA removal by astrocytic mitochondria induces lipid accumulation, followed by neurodegeneration that recapitulates key features of Alzheimer's disease (AD), including synaptic loss, neuroinflammation, demyelination, and cognitive impairment. Further, it was discovered that impaired FA degradation is an early event in the brain of a mouse model of AD. Findings suggest that increasing the activity of FA degradation in astrocytes, but not in neurons, may have better therapeutic benefits. Peroxisome proliferator-activated receptor α (PPARα) is a metabolic regulator of lipid metabolism, particularly in the degradation of excessive lipids in the form of free fatty acids. While the agonists of PPARα have shown protective effects in some animal experiments or pre-clinical studies of AD, the expression and function of PPARα in modulating metabolism and its potential adverse effects in treating neurodegenerative diseases, including AD, remain unclear and contradictory. Therefore, there is a pressing need for novel therapeutics and effective gene therapies to modulate PPARα expression precisely and delay or reverse the development of neurodegeneration by restoring astrocyte metabolic function. Thus, it is necessary to elucidate the cell type-specific role of PPARα in the brain and in the development of neurodegenerative diseases, as well as to develop precise therapeutic strategies that target PPARα and its downstream pathways with minimal adverse effects. These efforts could ultimately lead to efficacious treatments for patients with neurodegenerative diseases for which disease-modifying treatments are lacking.

### BRIEF SUMMARY OF THE INVENTION

It is an objective of the present invention to provide methods that allow for the treatment of neurodegenerative diseases by specifically upregulating the expression of PPARα (encoded by PPARA gene) in astrocytes, as specified in the independent claims. Embodiments of the invention are given in the dependent claims. Embodiments of the present invention can be freely combined with each other if they are not mutually exclusive.

In some embodiments, the present invention features a method of treating a neurodegenerative disease in a subject in need thereof. The method may comprise administering a viral vector comprising an astrocyte-specific promoter operatively linked to at least a portion of a PPARA gene to a subject diagnosed with a neurodegenerative disease. In some embodiments, the viral vector upregulates the expression of PPARA to promote lipid metabolism in the brain. In some embodiments, the viral vector comprises an astrocyte-specific promoter operatively linked to a full-length PPARA gene. In other embodiments, the viral vector comprises an astrocyte-specific promoter operatively linked to a fragment (e.g., a functional fragment) of a PPARA gene.

In other embodiments, the present invention features a method of delaying the onset of or preventing a neurodegenerative disease in a subject in need thereof. The method may comprise administering a viral vector comprising an astrocyte-specific promoter operatively linked to at least a portion of a PPARA gene to a subject at risk of having a neurodegenerative disease. In some embodiments, the viral vector upregulates the expression of PPARA to promote lipid metabolism in the brain. In some embodiments, the viral vector comprises an astrocyte-specific promoter operatively linked to a full-length PPARA gene. In other embodiments, the viral vector comprises an astrocyte-specific promoter operatively linked to a fragment (e.g., a functional fragment) of a PPARA gene.

Non-limiting examples of neurodegenerative disease may include but are not limited to Alzheimer's disease (AD), Parkinson's disease (PD) and other forms of Parkinsonism, Huntington's disease (HD), amyotrophic lateral sclerosis (ALS), multiple sclerosis (MS), frontotemporal dementia, Lewy body dementia, Gaucher's disease, progressive supranuclear palsy, stroke, depression, vascular dementia, and prion diseases.

One of the unique and inventive technical features of the present invention is the use of a viral vector, e.g., AAV-Astrocyte-PPARα, that increases the expression of PPARα in astrocytes. Without wishing to limit the invention to any theory or mechanism, it is believed that the technical feature of the present invention advantageously provides for targeted treatment of neurodegenerative disorders, e.g., AD. Without wishing to limit the present invention to any theory or mechanism, it is believed that the present invention advantageously provides (in SEQ ID NO: 1) an astrocyte-specific promoter operably linked to a nucleic acid encoding PPARα (including mouse or human variants, and also including functional variants or fragments), which enables astrocyte-selective upregulation of PPARα in the CNS. Within SEQ ID NO: 1, this promoter-PPARα cassette corresponds to: GFAP (short) promoter: nucleotides 158-838 and mouse Pparα coding sequence (RefSeq NM_011144.6): nucleotides 869-2272. Thus, the combined astrocyte-specific promoter-PPARα expression cassette spans approximately nucleotides 158-2272 of SEQ ID NO: 1. The intervening regulatory sequences located at positions 863-868, comprising a Kozak translation initiation sequence and the remaining elements (e.g., T2A, mCherry, WPRE, backbone sequences) serve supportive roles. None of the presently known prior references or works have the unique inventive technical feature of the present invention.

Moreover, the prior references teach away from the present invention. For example, current PPARα agonists lack cell selectivity, affecting all cells in the brain—including neurons, astrocytes, and other cell types—as well as other organs throughout the body. Specifically, targeting neurons may suppress pyruvate metabolism, which is critical for ATP production in the brain, and there are concerns about the potential carcinogenic effects of PPARα agonists on the liver.

In short, the present invention has resulted in the surprising finding that is taught away from by the priori references: that increased PPARα production and/or activity in astrocytes specifically is beneficial for the treatment of neurodegenerative disease, while, conversely, increasing PPARα production and/or activity in other cell types is often associated with negative side effects and/or null effects. Therefore, the ability of the present invention to target astrocytes specifically while leaving other cell types unaffected is believed to provide the present invention with a significant advantage over existing approaches. Furthermore, the prior references teach away from the present invention because increased PPARα production and/or activity, especially in peripheral tissues, is known to reduce bodyweight and/or Body Mass Index (BMI), and reduced bodyweight/BMI is a negative outcome associated with neurodegenerative disease. Furthermore, reduced bodyweight/BMI is not only associated with neurodegenerative disease, but is also believed to accelerate its progression, i.e., worsen it. Therefore, the present invention achieves a surprising result that is contrary to the teachings of the prior references, because by increasing PPARα production and/or activity, the present invention surprisingly (by targeting only astrocytes) prevents the loss of bodyweight, which is the exact opposite outcome when PPARα production and/or activity is increased in all cell types. Therefore, a person of ordinary skill in the art would be surprised to find that, contrary to the prior references, not only does increased PPARα production/activity actually not result in decreased BMI and accelerated progression of neurodegenerative disease, but that targeted increases in PPARα production and/or activity actually result in the opposite outcome: increased PPARα production and/or activity that is specifically targeted to astrocytes, especially those in the hypothalamus, actually increases BMI in patients with neurodegenerative disease, therefore slowing the acceleration or progression of said neurodegenerative disease.

Furthermore, the inventive technical features of the present invention contributed to a surprising result. For example, selectively upregulating PPARα protein expression in astrocytes can limit the progression of neurodegenerative diseases such as Alzheimer's disease (AD) while minimizing adverse effects on other cell types within the central nervous system and other organs.

In some embodiments, the present invention provides a method of using a viral vector, e.g., an adeno-associated viral (AAV) comprising an astrocyte-specific promoter operatively linked to at least a portion of a PPARA gene, e.g., AAV-Astrocyte-PPARα, for treating or preventing neurodegenerative diseases. For example, provided herein are methods for treating or preventing AD in a subject. In some embodiments, the method comprises administering to the subject a therapeutically effective amount of viral vectors that upregulates the expression of PPARα specifically inside astrocytes in the subject. More specifically, the methods disclosed herein are useful in modulating the expression of PPARα protein in astrocytes for the treatment of or to delay the development, onset, and symptoms related to neurodegenerative diseases. For example, the disclosed AAV-Astrocyte-PPARα is useful in the treatment of AD.

In one embodiment, the neurodegenerative disease is associated with protein aggregation, such as β-amyloid plaques. In another embodiment, neurodegenerative disease is associated with neuroinflammation, oxidative stress, and lipid dysregulation. In certain embodiments, the neurodegenerative disease is selected from the group consisting of Alzheimer's disease (AD), Parkinson's disease (PD), Huntington's disease (HD), amyotrophic lateral sclerosis (ALS), Huntington's disease (HD), frontotemporal dementia, Lewy Body dementia, stroke, depression, vascular dementia, and prion diseases.

Another aspect of the invention provides a method for improving neuronal health and brain function. In one embodiment, the AAV-Astrocyte-PPARα decreases the level of reactive oxygen species (ROS), diminishing the production of PLIN2 in brain tissues, and restoring the morphology and phenotype of astrocytes to non-disease conditions. In some embodiments, decreases the level of reactive oxygen species (ROS), diminishing the production of PLIN2 in brain tissues, and restoring the morphology and phenotype of astrocytes to non-disease conditions is measured relative to: a) an untreated subject diagnosed with the same neurodegenerative disease state; and/or b) the subject prior to the administration of the viral vector. In another embodiment, the AAV-Astrocyte-PPARα increases synaptic activity, improves short-term and long-term plasticity, and rescues the nest building and the novel object recognition behavior compared to those in AD subjects. In some embodiments, increasing synaptic activity, improved short-term and long-term plasticity, and rescue of the nest building and the novel object recognition behavior is measured relative to a) an untreated subject diagnosed with the same neurodegenerative disease state; and/or b) the subject prior to the administration of the viral vector.

The subject may be a human subject, for example, a human subject exhibiting symptoms of AD and dementia. The composition may be administered by intravenous injection with appropriate AAV vectors. Alternatively, the composition is administered by an intranasal delivery, direct injection into the brain (including through a small hole in the skull or using a catheter; including but not limited to direct injection into the hippocampus and/or hypothalamus), implantable devices, nanoparticle-based delivery systems (to transport drugs across the blood-brain barrier), and the like.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows the map of the design of the AAV5-GFAP-PPARα construct in mouse (top) and human (bottom) isoforms.

FIG. 2 shows the expression of PPARα in astrocytes in the 5×FAD mouse brain which are injected with AAV5-GFAP-mPpara-mCherry-WPRE. mCherry expression (a reporter protein co-expressed with PPARα), GFAP expression, DAPI levels, and a merged image are shown. 88.5% of the GFAP positive cells are also expressing reporter protein. Scale bar, 50 μm.

FIGS. 3A-3C shows AAV5-GFAP-PPARα injection to the hippocampus improves fEPSP and long-term potentiation (LTP) in 5×FAD mice. FIG. 3A shows a brightfield photomicrograph of a hippocampal slice on a MED64 electrode array. FIG. 3B shows a time course for Theta-burst induced LTP. FIG. 3C shows the mean LTP 35-40 min after Theta-burst. Closed circle, AAV5-GFAP-Reporter (without mPpara) treated wildtype (WT); triangle, AAV5-GFAP-Reporter treated 5×FAD (5×FAD); square, AAV5-GFAP-PPARα treated 5×FAD mice (5×FAD-PPARα). n=3 mice for each group. **p<0.01, ***p<0.001.

FIGS. 4A-4B show AAV5-GFAP-PPARα injection to the hippocampus rescues the short-term plasticity (STP) in 5×FAD mice. FIG. 4A shows the mean STP 15-20 min after Theta-burst, ***p<0.001. FIG. 4B shows paired-pulse ratio (PPR) measured at different inter-stimulus intervals (30, 40, and 50 ms). Closed circle, WT; triangle, 5×FAD; square, 5×FAD-PPARα. n=3 mice for each group. **WT vs. 5×FAD, ##5×FAD vs. 5×FAD-PPARα. *p<0.05; ** or ##p<0.01.

FIGS. 5A-5B show AAV5-GFAP-PPARα injection to the hippocampus alleviates behavioral deficits in 5×FAD mice. FIG. 5A shows results from nest building tests, including nest scores and unshredded nestle weight. FIG. 5B. shows the discrimination index by novel object recognition (NOR) test. Closed circle, WT; triangle, 5×FAD; square, 5×FAD-PPARα. n=4-6 mice for each group. *p<0.05.

FIG. 6 shows immunostaining results of perilipin-2 (PLIN2) from the hippocampal region of WT, 5×FAD, and 5×FAD-PPARα mice. Both WT and 5×FAD mice were injected with control AAV, carrying reporter genes but not PPARα. 5×FAD-PPARα mice were injected with experimental AAV (carrying PPARα) and therefore experienced induced astrocyte-specific PPARα upregulation in the hippocampus. Left: PLIN2 and DAPI single-channel images and merged images are shown. Right: quantification of the PLIN2 expression. Scale bar, 50 μm. Closed circle, WT; triangle, 5×FAD; square, 5×FAD-PPARα. n=4 mice for each group. *p<0.05, **p<0.01.

FIG. 7 shows immunostaining results of 4-Hydroxynonenal (4-HNE; a marker for lipid peroxidation and oxidative stress) from the hippocampal region of WT, 5×FAD, and 5×FAD-PPARα mice. Both WT and 5×FAD mice were injected with control AAV, carrying reporter genes but not PPARα. 5×FAD-PPARα mice were injected with experimental AAV (carrying PPARα) and therefore experienced induced astrocyte-specific PPARα upregulation in the hippocampus. Left: 4-HNE and DAPI single-channel images and merged images. Right: quantification of the 4-HNE levels. Scale bar, 50 μm. Closed circle, WT; triangle, 5×FAD; square, 5×FAD-PPARα. n=4 mice for each group. *p<0.05, **p<0.01.

FIG. 8 shows immunostaining results of amyloid antibody 6E10 from the hippocampal region of WT, 5×FAD, and 5×FAD-PPARα mice. Both WT and 5×FAD mice were injected with control AAV, carrying reporter genes but not PPARα. 5×FAD-PPARα mice were injected with experimental AAV (carrying PPARα) and therefore experienced induced astrocyte-specific PPARα upregulation in the hippocampus. Left: 6E10 and DAPI single-channel images and merged images. Right: quantification of the amyloid levels. Scale bar, 50 μm. Closed circle, WT; triangle, 5×FAD; square, 5×FAD-PPARα. n=4 mice for each group. **p<0.01, ***p<0.001.

FIG. 9 shows immunostaining results of GFAP from the hippocampal region of WT, 5×FAD, and 5×FAD-PPARα mice. Both WT and 5×FAD mice were injected with control AAV, carrying reporter genes but not PPARα. 5×FAD-PPARα mice were injected with experimental AAV (carrying PPARα) and therefore experienced induced astrocyte-specific PPARα upregulation in the hippocampus. Left: GFAP and DAPI single-channel images and merged images. Right: quantification of the length of astrocyte processes including primary branches and secondary branches. Each dot indicates the average branch length of one individual cell from 4 mice per group. Scale bar, 50 μm. Closed circle, WT; triangle, 5×FAD; square, 5×FAD-PPARα. *p<0.05, **p<0.01, ***p<0.001.

FIGS. 10A-10B shows nest-building behavior assessed in WT, 5×FAD, and 5×FAD-PPARα mice. Both WT and 5×FAD mice were injected with control AAV, carrying reporter genes but not PPARα. 5×FAD-PPARα mice were injected with experimental AAV (carrying PPARα) and therefore experienced induced astrocyte-specific PPARα upregulation in the hypothalamus. FIG. 10A shows nest-building score evaluated using a standardized scoring system. FIG. 10B shows unshredded nestlet weight measured at the end of the testing period. Closed circle, WT; square, 5×FAD; triangle, 5×FAD-PPARα. n=4-6 mice per group. *p<0.05, **p<0.01.

FIGS. 11A-11F shows the effects of astrocyte-specific PPARα upregulation in the hypothalamus on body weight and body composition in WT, 5×FAD, and 5×FAD-PPARα mice. FIG. 11A shows body weight measured longitudinally over the experimental period. FIG. 11B shows body weight gain relative to baseline. FIG. 11C shows fat mass measured by EchoMRI.

FIG. 11D shows fat mass normalized to body weight (Fat/BW). FIG. 11E shows lean mass measured by EchoMRI. FIG. 11F shows lean mass normalized to body weight (Lean/BW). Closed circle, WT; square, 5×FAD; triangle, 5×FAD-PPARα. n=4-6 mice per group. *p<0.05, **p<0.01, ***p<0.001.

FIG. 12 shows relative mRNA expression levels of genes associated with lipid metabolism, sympathetic nervous system signaling, and thermogenesis (Acc, Ppara, Atgl, B2, B3r, Ucp1, and Cox8b) in inguinal white adipose tissue (iWAT) from WT, 5×FAD, and 5×FAD-PPARα mice following astrocyte-specific PPARα upregulation in the hypothalamus. Expression of Acc, Ppara, Atgl, B2r, B3r, Ucp1, and Cox8b was quantified by RT-PCR and normalized to housekeeping gene expression. Data are presented relative to WT controls. Closed circle, WT; square, 5×FAD; triangle, 5×FAD-PPARα. n=4-6 mice per group. *p<0.05, **p<0.01, ***p<0.001.

FIGS. 13A-13B show information regarding a viral vector, here, an AAV-Astrocyte-PPARα, which increases the expression of PPARα in astrocytes. FIG. 13A shows a vector map of said viral vector. FIG. 13B shows a vector summary of said vector.

FIGS. 14A-14C show further information regarding a viral vector, (here, an AAV-Astrocyte-PPARα, which increases the expression of PPARα in astrocytes), including its constituent components. FIG. 14A shows information regarding components of the viral vector, starting at the vector's 5′ end. FIG. 14B shows further information regarding further components of the viral vector. FIG. 14C shows information regarding experimental validation of said vector by restriction enzyme digestion.

### DETAILED DESCRIPTION OF THE INVENTION

Disclosed are the various compounds, solvents, solutions, carriers, and/or components to be used to prepare the compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, while specific reference of each various individual and collective combination and permutation of these compounds may not be explicitly disclosed, each is specifically contemplated and described herein.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human.

As used herein, the terms “treat,” “treating,” or “treatment” refer to both therapeutic treatment and prophylactic or preventative measures, with the objective of preventing, reducing, slowing down (lessen), inhibiting, or eliminating an undesired physiological change, symptom, disease, or disorder. For example, the disease may be a neurodegenerative disease. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition prior to administration of the compositions of the invention. Subjects at risk for the disease can be identified by, for example, any or a combination of appropriate diagnostic or prognostic assays known in the art.

As used herein, “clinical improvement” may refer to a noticeable reduction in the symptoms of a disorder, or cessation thereof.

The terms “manage,” “managing,” and “management” refer to preventing or slowing the progression, spread, or worsening of a disease or disorder, or of one or more symptoms thereof. In certain cases, the beneficial effects that a subject derives from a prophylactic or therapeutic agent do not result in a cure of the disease or disorder.

The terms “administering” and “administration” refer to methods of providing a pharmaceutical preparation, composition, or formulation to a subject. The compositions described herein can be administered in a number of ways depending on whether local or systemic treatment is desired, and on the area to be treated. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single-dose compositions can contain such amounts or submultiples thereof to make up the daily dose. The dosage can be adjusted by the individual physician in the event of any contraindications. Dosage can vary, and can be administered in one or more dose administrations daily, for one or several days, weekly, twice weekly, etc. Guidance can be found in the literature for appropriate dosages for given classes of pharmaceutical products.

The exact amount of the compositions required will vary from subject to subject, depending on the species, age, weight, and general condition of the subject, the severity of the disorder being treated, the particular composition used, its mode of administration, and the like. Thus, it is not possible to specify an exact amount for every composition. However, an appropriate amount can be determined by one of ordinary skill in the art using only routine experimentation given the teachings herein.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

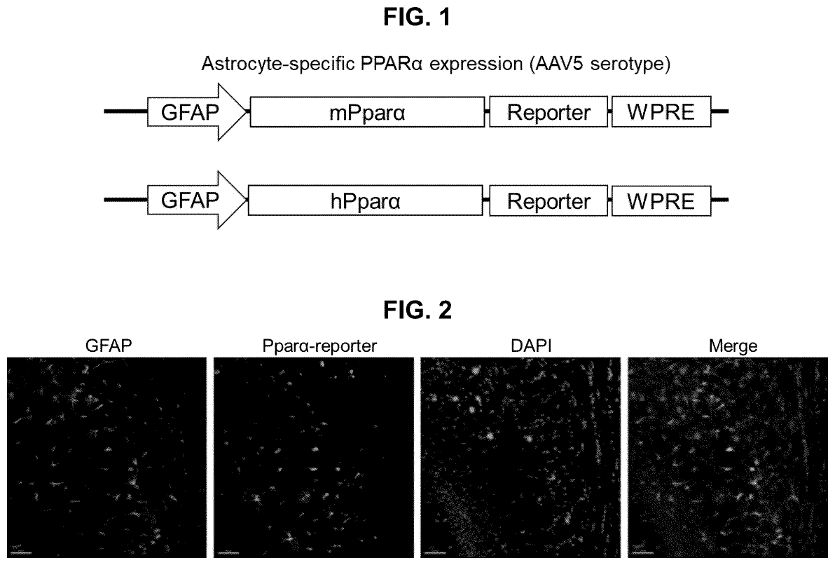

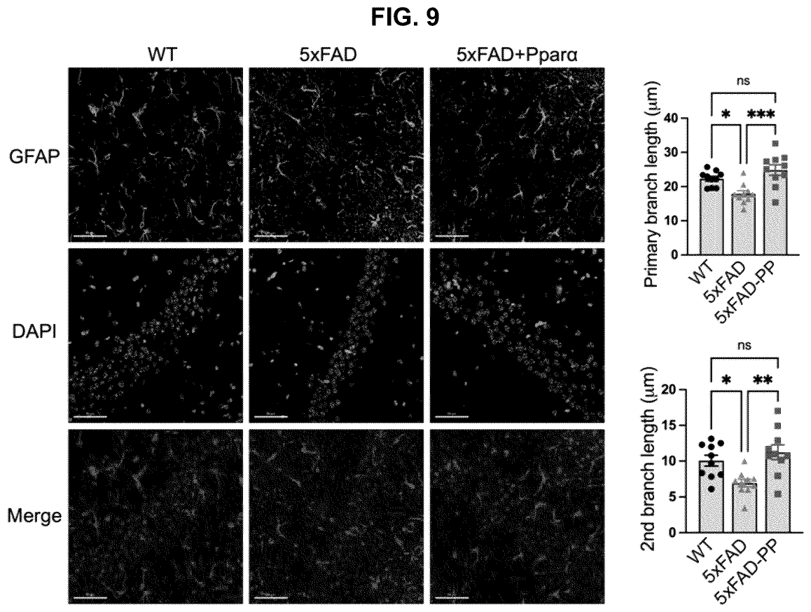

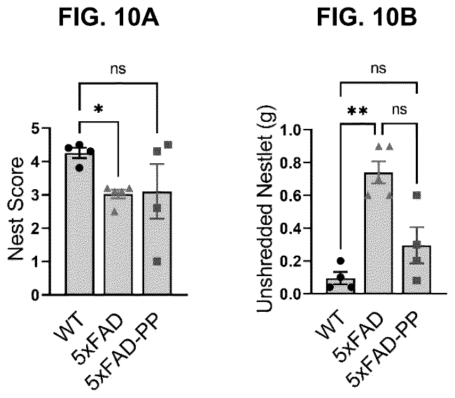

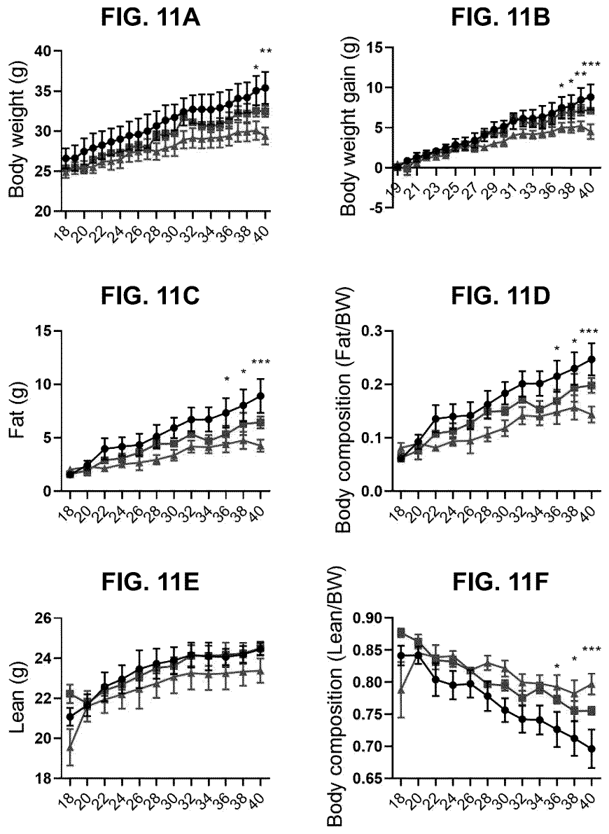

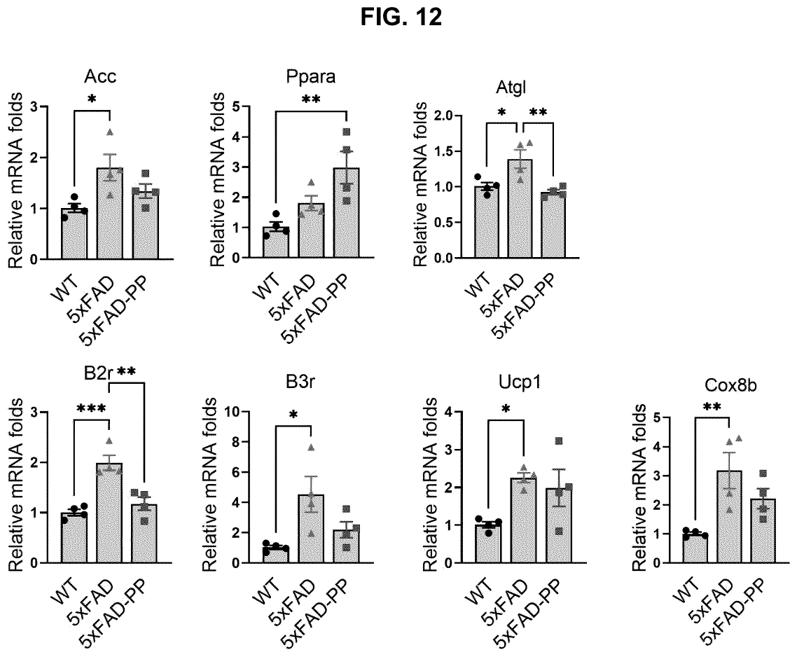

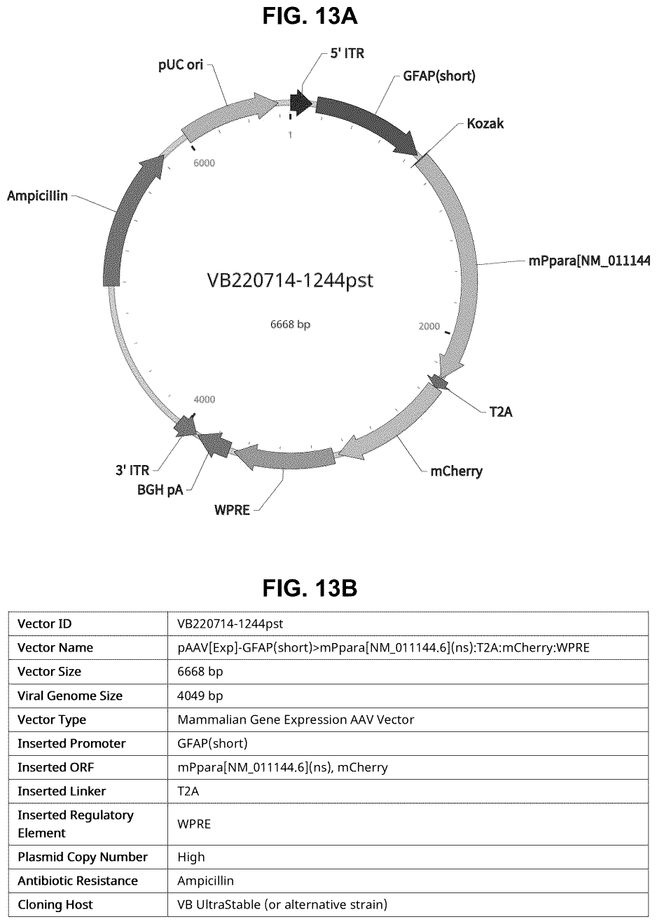

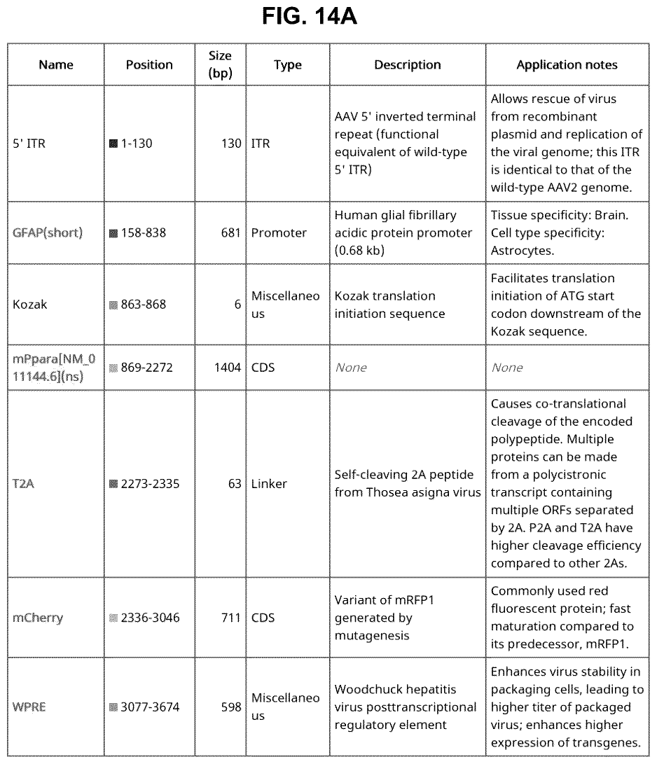

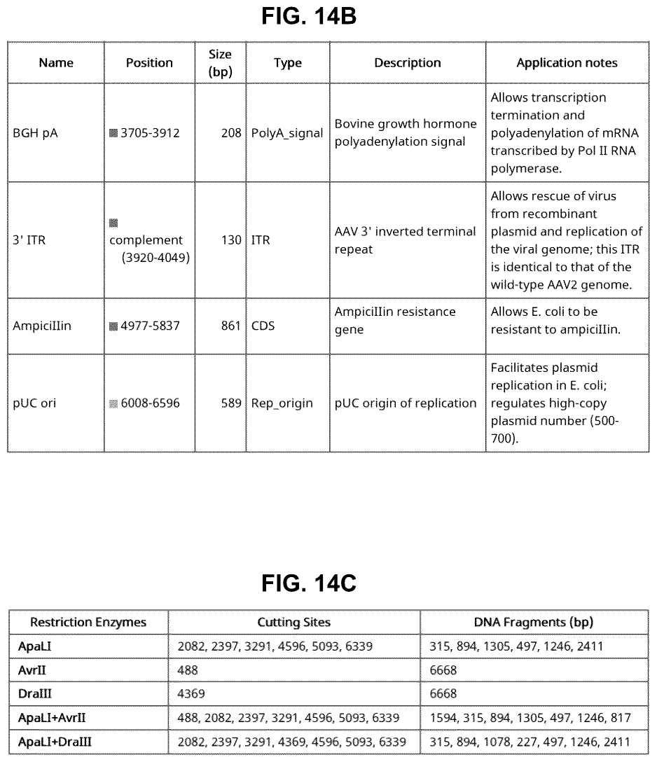

Referring now to FIGS. 1-14C, the present invention features methods and compositions for manipulating metabolism in astrocytes to improve astrocytic and neuronal function, as well as individual pathology hallmarks and symptoms during the development of neurodegeneration. In particular, it relates to increasing PPARα expression and/or activity, specifically in astrocytes within the central nervous system, to treat a variety of neurodegenerative conditions, specifically those that cause neuronal lipid dysregulation, oxidative damages, accumulation of β-amyloid or other protein aggregates, dementia, and motor dysfunction.

The present invention features methods of treating a neurodegenerative disease in a subject in need thereof. In some embodiments, the method comprises administering a viral vector comprising an astrocyte-specific promoter operatively linked to at least a portion of a PPARA gene to a subject diagnosed with a neurodegenerative disease. In some embodiments, the viral vector upregulates the expression of PPARA to promote lipid metabolism in the brain. In some embodiments, the viral vector comprises an astrocyte-specific promoter operatively linked to a full-length PPARA gene. In other embodiments, the viral vector comprises an astrocyte-specific promoter operatively linked to a fragment (e.g., a functional fragment) of a PPARA gene.

The present invention may also feature methods of delaying the onset of or preventing a neurodegenerative disease in a subject in need thereof. In some embodiments, the method comprises administering a viral vector comprising an astrocyte-specific promoter operatively linked to at least a portion PPARA gene to a subject at risk of having a neurodegenerative disease. In some embodiments, the viral vector upregulates the expression of PPARA to promote lipid metabolism in the brain. In some embodiments, the viral vector comprises an astrocyte-specific promoter operatively linked to a full-length PPARA gene. In other embodiments, the viral vector comprises an astrocyte-specific promoter operatively linked to a fragment (e.g., a functional fragment) of a PPARA gene

In some embodiments, the present invention features a method of preventing or treating a neurodegenerative disease in a subject in need thereof, the method comprising: (a) identifying the subject presenting with the neurodegenerative disease; and (b) administering a viral vector with an astrocyte-specific promoter that carries and upregulates expression of PPARA gene to promote lipid metabolism. In some embodiments, identifying a subject presenting a neurodegenerative disease involves clinically diagnosing the subject with at least one of the neurodegenerative diseases mentioned herein.

Examples of neurodegenerative diseases that may be treated or prevented using the methods and viral vectors described herein include, but are not limited to, the following: Alzheimer's disease (AD), Parkinson's disease (PD) and other forms of Parkinsonism, Huntington's disease (HD), amyotrophic lateral sclerosis (ALS), multiple sclerosis (MS), frontotemporal dementia, Lewy body dementia, Gaucher's disease, progressive supranuclear palsy, stroke, depression, vascular dementia, and prion diseases. Non-limiting examples of prion disease may include but are not limited to Creutzfeldt-Jakob disease and variant Creutzfeldt-Jakob disease.

In some embodiments, the viral vector is an adeno-associated viral (AAV) vector. In other embodiments, the viral vector is a lentiviral vector. Non-limiting examples of AAV vectors include but are not limited to AAV2, AAV5, AAV6, AAV9, AAV-PHP.eB, AAV-PHP.B, or AAVrh10 serotypes. Other efficient gene delivery and selective transduction systems may also be employed in the methods described herein.

In some embodiments, the astrocyte-specific promoter is a Glial Fibrillary Acidic Protein (GFAP) promoter. In some embodiments, the astrocyte-specific promoter is a human GFAP promoter, e.g., a 2.2 kb human GFAP promoter (e.g., gfa2). In other embodiments, the astrocyte-specific promoter is truncated GFAP promoter (e.g., GfaABC1D, 0.7 kb). In some embodiments, the astrocyte-specific promoter is a gfa2 (2.2 kb), gfa2 (B) 3 (2.6 kb), or gfa2 (ABD) 3. In accordance with the viral vectors described herein, other astrocyte-specific promoters may be utilized. Additionally, truncated or variant forms of these promoters may be employed, including, but not limited to, human or rat ALDH1L1 (0.9-2.1 kb), mouse Slc1a3 (Glast) (0.64 kb), and human GJB3 (Cx30) (0.5 kb).

In some embodiments, the viral vector may be administered intravenously, intranasally, intrathecally, intracisternally, intracerebroventricularly, or a combination thereof. In some embodiments, the viral vector may be administered via intravenous injection, subcutaneous injection, intramuscular injection, or a combination thereof. In other embodiments, the viral vector may be administered via intranasal delivery, intrathecal delivery, or a combination thereof. In other embodiments, the viral vector may be administered directly into the brain, such as through a small hole in the skull, using a catheter, or via intraparenchymal or intracerebral injection, including but not limited to direct administration to the hippocampus and/or hypothalamus. In further embodiments, the viral vector may be administered via a nanoparticle delivery system. Regardless of the administration method, the viral vectors described herein are capable of crossing the blood-brain barrier.

In certain embodiments, the viral vector is administered intravenously. Thus, without wishing to limit the present invention to any theory or mechanism, it is believed that for intravenous (IV) administration targeting brain delivery, the viral vector must exhibit enhanced blood-brain barrier penetrability. In some embodiments, this requirement may be achieved through the use of specific AAV serotypes, such as AAV-PHP.eB, AAV-PHP.B, AAVrh10, and AAV9, or through other modifications of existing AAV serotypes.

Thus, in some embodiments, the present invention may feature methods of treating a neurodegenerative disease in a subject in need thereof. In some embodiments, the method comprises administering an AAV vector comprising an astrocyte-specific promoter (e.g., a GFAP promoter) operatively linked to at least a portion of a PPARA gene to a subject diagnosed with a neurodegenerative disease. In other embodiments, the method comprises intravenously administering an AAV vector comprising an astrocyte-specific promoter (e.g., a GFAP promoter) operatively linked to at least a portion of a PPARA gene to a subject diagnosed with a neurodegenerative disease.

In other embodiments, the present invention may feature methods of delaying the onset of a neurodegenerative disease in a subject in need thereof. In some embodiments, the method comprises administering an AAV vector comprising an astrocyte-specific promoter (e.g., a GFAP promoter) operatively linked to at least a portion of a PPARA gene to a subject diagnosed with a neurodegenerative disease. In other embodiments, the method comprises intravenously administering an AAV vector comprising an astrocyte-specific promoter (e.g., a GFAP promoter) operatively linked to at least a portion of a PPARA gene to a subject diagnosed with a neurodegenerative disease.

In some embodiments, a therapeutically effective dose of the viral vector is administered to treat (or prevent) the neurodegenerative disease. The precise amount required will vary depending on the species, age, weight, and general condition of the subject, as well as the severity of the disorder being treated, the specific viral vector used, and its mode of administration. Therefore, it is not feasible to specify an exact dosage for every situation. However, one of ordinary skill in the art can determine the appropriate amount through routine experimentation, given the teachings herein.

In some embodiments, the present invention features a method of delaying the onset of, treating, or preventing a neurodegenerative disease in a subject in need thereof, comprising administering to the subject a therapeutically effective amount of a viral vector comprising a nucleic acid molecule encoding for one or more homologous genes encoding PPARα (e.g., a functional isoform or functional fragment) which upregulate the expression and/or activity of PPARα selectively inside of astrocytes in the central nervous system. In some embodiments, the viral vector further comprises an astrocytes-specific promoter operatively linked to the PPARα nucleic acid molecule. As used herein, “homologous genes” may refer to genes that share a common evolutionary origin with the human PPARA gene but from a different species (e.g., mouse Ppara gene).

In some embodiments, the nucleic acid molecule encoding PPARα for upregulating the expression of PPARα and its functional fragments or isoforms. As used herein, a ‘functional fragment’ or ‘functional isoform’ refers to a nucleic acid that encodes a polypeptide capable of functioning, such as a transcription factor, in a subject, similar to the full-length nucleic acid encoding PPARα. In the case of PPARα more specifically, a “functional fragment” may include a portion of the protein comprising at least one functional domain selected from the DNA-binding domain (C domain), ligand-binding domain (E domain), activation function domain (AF-1 or AF-2), hinge region containing nuclear localization sequences, or combinations thereof, provided that the fragment retains measurable transcriptional regulatory activity, such as binding to a PPAR response element (PPRE), heterodimerization with RXR, recruitment of transcriptional co-regulators, or modulation of expression of one or more PPARα targeting genes.

In some embodiments, the astrocyte-specific promoter is selected from the nucleic acid molecule consisting of different forms of GFAP promoter (human gfa2, 2.2 kb; GfaABC1D, 0.7 kb; gfa2 (B) 3, 2.6 kb; gfa28, 0.45 kb) or other astrocyte-specific promoters and their truncates or variants (human/rat ALDH1L1, 0.9-2.1 kb; mouse Slc1a3 (Glast), 0.64 kb; human GJB3 (Cx30), 0.5 kb).

In some embodiments, the viral vector is an adeno-associated viral (AAV) vector. In other embodiments, the viral vector is a lentiviral vector. Non-limiting examples of AAV vectors include but are not limited to AAV2, AAV5, AAV6, AAV9, AAV-PHP.eB, AAV-PHP.B, or AAVrh10 serotypes.

In certain embodiments, methods described herein, e.g., administering a therapeutically effective dose of a viral vector, e.g., AAV-Astrocyte-PPARα, reduce the levels of reactive oxygen species (ROS) and oxidative stress in brain tissues affected by neurodegenerative diseases such as Alzheimer's disease (AD). Oxidative stress, primarily caused by the generation of ROS, is a hallmark of AD, leading to damage to proteins, lipids, and DNA, as well as inflammation and cell death.

In some embodiments, the methods described herein, such as administering a therapeutically effective dose of a viral vector like AAV-Astrocyte-PPARα, reduce the accumulation of neutral lipid species in brain tissues affected by neurodegenerative diseases such as Alzheimer's disease (AD). The accumulation of neutral lipids in the form of lipid droplets is a hallmark of AD brains and is associated with disease progression.

In certain embodiments, the methods described herein, such as administering a therapeutically effective dose of a viral vector like AAV-Astrocyte-PPARα, increases synaptic function, including improvements in both short-term and long-term plasticity in the subject. Additionally, in some embodiments, the methods described herein restore cognitive function, including cognitive and spatial memory, in the subject through the therapeutically effective dose of AAV-Astrocyte-PPARα. In some embodiments, increased synaptic function, including improvements in both short-term and long-term plasticity in the subject, is measured relative to: a) an untreated subject diagnosed with the same neurodegenerative disease state; and/or b) the subject prior to the administration of the viral vector. In some embodiments, restoration of cognitive function, including cognitive and spatial memory, is measured relative to: a) an untreated subject diagnosed with the same neurodegenerative disease state; and/or b) the subject prior to the administration of the viral vector.

In some embodiments, methods described herein alleviate Alzheimer's disease-associated astrocyte reactivity and morphological features in the subject.

In some embodiments, the upregulation of PPARα can be effectuated by administering a small molecule that selectively targets astrocytes and promotes PPARα expression and/or activity. Such small molecules can be identified by routine drug screening protocols. For example, to screen for a PPARα agonist, a cell line such as HepG2 can be engineered to express a reporter gene, like luciferase, under the control of a PPARα-responsive promoter. After treating the cells with potential agonist compounds, the activity of the reporter gene (e.g., luciferase luminescence) is measured. In some embodiments, an increase in reporter activity compared to a vehicle-treated control group indicates activation of PPARα by the tested compound, suggesting it may be a potential agonist. The screening methods described herein are well-established for identifying PPARα agonists, and variations of these methods may be utilized in accordance with the present invention. In certain embodiments, the cell lines used for the aforementioned screening methods may include HepG2, HEK293, HepaRG, or C2C12. Non-limiting examples of reporter genes or reporters may include luciferase, green fluorescent protein (GFP), β-Galactosidase (LacZ), or mCherry.

The present invention may also feature a viral vector comprising an astrocyte-specific promoter operatively linked to at least a portion of a PPARA gene for use in a method of treating a neurodegenerative disease in a subject diagnosed with a neurodegenerative disease. In some embodiments, the viral vector upregulates the expression of PPARA to promote lipid metabolism in the brain. In some embodiments, the viral vector comprises an astrocyte-specific promoter operatively linked to a full-length PPARA gene. In other embodiments, the viral vector comprises an astrocyte-specific promoter operatively linked to a fragment (e.g., a functional fragment) of a PPARA gene.

In other embodiments, the present invention may feature a viral vector comprising an astrocyte-specific promoter operatively linked to at least a portion of a PPARA gene for use in a method of preventing a neurodegenerative disease in a subject at risk of having a neurodegenerative disease. In some embodiments, the viral vector upregulates the expression of PPARA to promote lipid metabolism in the brain. In some embodiments, the viral vector comprises an astrocyte-specific promoter operatively linked to a full-length PPARA gene. In other embodiments, the viral vector comprises an astrocyte-specific promoter operatively linked to a fragment (e.g., a functional fragment) of a PPARA gene.

The present invention may further feature the use of a viral vector comprising an astrocyte-specific promoter operatively linked to at least a portion of a PPARA gene in the manufacture of a medicament for the treatment of a neurodegenerative disease. In some embodiments, the viral vector comprises an astrocyte-specific promoter operatively linked to a full-length PPARA gene. In other embodiments, the viral vector comprises an astrocyte-specific promoter operatively linked to a fragment (e.g., a functional fragment) of a PPARA gene.

In some embodiments, the present invention may feature a method of treating or delaying the onset of or preventing a neurodegenerative disease in a subject in need thereof, the method comprising administering a viral vector comprising an astrocyte-specific promoter operatively linked to at least a portion of a PPARA gene to a subject, wherein the viral vector upregulates the expression of PPARA to promote lipid metabolism selectively inside of astrocytes in the central nervous system. In some embodiments, the subject may be diagnosed with a neurodegenerative disease. In some embodiments, the subject may not yet be diagnosed with a neurodegenerative disease. In some embodiments, the subject may have a high risk of developing a neurodegenerative disease. In some embodiments, the subject may be in a prodromal stage of a neurodegenerative disease.

In some embodiments, the neurodegenerative disease is selected from a group consisting of Alzheimer's disease (AD), Parkinson's disease (PD), Huntington's disease (HD), amyotrophic lateral sclerosis (ALS), multiple sclerosis (MS), frontotemporal dementia, Lewy body dementia, Gaucher's disease, progressive supranuclear palsy, stroke, depression, vascular dementia, and prion diseases.

In some embodiments, the viral vector is an adeno-associated viral (AAV) vector or a lentiviral vector. In some embodiments, the lentiviral vector is selected from a group consisting of AAV2, AAV5, AAV6, AAV9, AAV-PHP.eB, AAV-PHP.B, or AAVrh10 serotypes.

In some embodiments, the viral vector comprises an astrocyte-specific promoter operatively linked to at least a fragment of a PPARA gene. In some embodiments, the astrocyte-specific promoter is a Glial Fibrillary Acidic Protein (GFAP) promoter.

In some embodiments, the viral vector is administered intravenously, intranasally, intrathecally, intracisternally, intracerebroventricularly, or a combination thereof. In some embodiments, the viral vector is administered via direct injection into the brain. In some embodiments, the viral vector is administered via direct injection to the hippocampus and/or hypothalamus and/or other region(s) or structure(s) of the brain. In some embodiments, administration of the viral vector via direct injection to the hypothalamus may result in increased PPARα production and/or activity specifically targeted to astrocytes in the hypothalamus, which may cause increased BMI in patients with neurodegenerative disease, therefore slowing the acceleration or progression of said neurodegenerative disease that is associated with decreased BMI.

In some embodiments, the viral vector is administered via a nanoparticle delivery system. In some embodiments, the viral vector is transported across the blood-brain barrier.

In some embodiments, the viral vector decreases the level of reactive oxygen species (ROS) and oxidative stresses in brain tissues affected by the neurodegenerative diseases, relative to: a) an untreated subject diagnosed with the same neurodegenerative disease state; and/or b) the subject prior to the administration of the viral vector. In some embodiments, the viral vector diminishes the accumulation of neutral lipid species in brain tissues affected by the neurodegenerative disease, relative to: a) an untreated subject diagnosed with the same neurodegenerative disease state; and/or b) the subject prior to the administration of the viral vector.

In some embodiments, the viral vector increases synaptic function, wherein synaptic function includes short-term and long-term plasticity. In some embodiments, the viral vector rescues cognitive function; wherein cognitive function includes cognitive memory and spatial memory. In some embodiments, increases in synaptic function and rescue of cognitive function are measured relative to: a) an untreated subject diagnosed with the same neurodegenerative disease state; and/or b) the subject prior to the administration of the viral vector.

In some embodiments, the viral vector comprises a nucleotide sequence with at least 90% sequence identity to SEQ ID NO: 1, SEQ ID NO: 3, or SEQ ID NO: 7.

In some embodiments, the viral vector comprises a nucleotide sequence with at least 90% sequence identity to a segment of SEQ ID NO: 1 beginning at nucleotide 869 and ending at nucleotide 2272.

In other embodiments, the present invention features a viral vector comprising an astrocyte-specific promoter operatively linked to at least a portion of a PPARA gene for use in a method of treating or delaying the onset of or preventing a neurodegenerative disease in a subject diagnosed with a neurodegenerative disease, wherein the viral vector upregulates the expression of PPARA to promote lipid metabolism selectively inside of astrocytes in the central nervous system. In some embodiments, the viral vector comprises the polynucleotide of SEQ ID NO: 1.

In other embodiments, the present invention features a method of treating peripheral metabolic dysregulation associated with neurodegenerative disease in a subject in need thereof, the method comprising administering a viral vector comprising an astrocyte-specific promoter operatively linked to at least a portion of a PPARA gene to a hypothalamus of a subject diagnosed with a neurodegenerative disease, wherein the viral vector upregulates the expression of PPARA to promote lipid metabolism in the hypothalamus of the subject, thereby indirectly altering downstream lipid metabolism in at least a peripheral portion of the subject.

In some embodiments, the viral vector is an adeno-associated viral (AAV) vector or a lentiviral vector. In some embodiments, the viral vector comprises the polynucleotide of SEQ ID NO: 1.

In some embodiments, the viral vector comprises the polynucleotide of SEQ ID NO: 1. In some embodiments, SEQ ID NO: 1 comprises AAV Vector-pAAV[Exp]-GFAP(short)>mPpara[NM_011144.6] (ns): T2A: mCherry: WPRE. In some embodiments, SEQ ID NO: 1 comprises an adeno-associated virus (AAV) expression construct designated pAAV [Exp]-GFAP(short)>mPparaNM_011144.6: T2A: mCherry: WPRE. In some embodiments, the construct comprises (i) a shortened human glial fibrillary acidic protein (GFAP) promoter configured to drive astrocyte-specific expression; (ii) a nucleic acid sequence encoding mouse Ppara (mPpara), corresponding to RefSeq accession number NM_011144.6; (iii) a Thosea asigna virus 2A (T2A) self-cleaving peptide sequence operably linking mPpara and mCherry to permit bicistronic expression; (iv) a nucleic acid sequence encoding the fluorescent reporter protein mCherry; and (v) a woodchuck hepatitis virus posttranscriptional regulatory element (WPRE) to enhance transcript stability and expression.

In some embodiments, the present invention may also utilize, in various capacities, SEQ ID NO: 2, SEQ ID NO: 3, SEQ ID NO: 4, SEQ ID NO: 5, SEQ ID NO: 6, and SEQ ID NO: 7. SEQ ID NO: 2 comprises Human glial fibrillary acidic protein promoter short (0.68 kb). SEQ ID NO: 3 comprises mPpara[NM_011144.6] (ns). In this context, [NM_011144.6] refers to Mus musculus peroxisome proliferator activated receptor alpha (Ppara), transcript variant 1. SEQ ID NO: 4 comprises T2A-Self-cleaving 2A peptide from Thosea asigna virus. SEQ ID NO: 5 comprises mCherry. SEQ ID NO: 6 comprises WPRE—Woodchuck hepatitis virus posttranscriptional regulatory element. SEQ ID NO: 7 comprises Homo sapiens peroxisome proliferator activated receptor alpha (PPARA), transcript variant 5.

Further included in the present invention are nucleic acid sequences which are greater than 85%, preferably at least about 90%, more preferably at least about 95%, more preferably still about 97%, and most preferably at least about 98 to 99% identical or homologous to the sequences of the invention, including those in the Sequence Listing [SEQ ID NO:1; SEQ ID NO: 2, SEQ ID NO: 3, SEQ ID NO: 4, SEQ ID NO: 5, SEQ ID NO: 6, and SEQ ID NO: 7].

In some embodiments, the present invention may feature a method of treating peripheral metabolic dysregulation associated with neurodegenerative disease in a subject in need thereof, the method comprising administering a viral vector comprising an astrocyte-specific promoter operatively linked to at least a portion of a PPARA gene to a hypothalamus of a subject diagnosed with a neurodegenerative disease, wherein the viral vector upregulates the expression of PPARA to promote lipid metabolism in the hypothalamus of the subject, thereby indirectly altering downstream lipid metabolism in at least a peripheral portion of the subject.

In some embodiments, the viral vector comprises an astrocyte-specific promoter operatively linked to at least a fragment of a PPARA gene.

In some embodiments, the viral vector comprises a nucleotide sequence with at least 90% sequence identity to SEQ ID NO: 1, SEQ ID NO: 3, or SEQ ID NO: 7.

In some embodiments, the viral vector comprises a nucleotide sequence with at least 90% sequence identity to a segment of SEQ ID NO: 1 beginning at nucleotide 869 and ending at nucleotide 2272.

In some embodiments, the present invention may feature a method of delaying the onset of or preventing peripheral metabolic dysregulation associated with a neurodegenerative disease in a subject in need thereof, the method comprising administering a viral vector comprising an astrocyte-specific promoter operatively linked to at least a portion PPARA gene to a hypothalamus of subject at risk of having a neurodegenerative disease, wherein the viral vector upregulates the expression of PPARA to promote lipid metabolism in the hypothalamus of the subject, thereby indirectly altering downstream lipid metabolism in at least a peripheral portion of the subject.

In some embodiments, the present invention may feature a viral vector comprising an astrocyte-specific promoter operatively linked to at least a portion of a PPARA gene for use in a method of treating peripheral metabolic dysregulation associated with neurodegenerative disease in a subject diagnosed with a neurodegenerative disease, wherein the viral vector upregulates the expression of PPARA to promote lipid metabolism in a hypothalamus of the subject, thereby indirectly altering downstream lipid metabolism in at least a peripheral portion of the subject.

In some embodiments, the present invention may feature a viral vector comprising an astrocyte-specific promoter operatively linked to at least a portion of a PPARA gene for use in a method of preventing peripheral metabolic dysregulation associated with neurodegenerative disease in a subject diagnosed with a neurodegenerative disease, wherein the viral vector upregulates the expression of PPARA to promote lipid metabolism in a hypothalamus of the subject, thereby indirectly altering downstream lipid metabolism in at least a peripheral portion of the subject.

In some embodiments, the present invention may feature A hypothalamus comprising genetically-modified hypothalamic astrocyte cells, the genetically modified hypothalamic cells comprising a viral vector, the viral vector comprising an astrocyte-specific promoter operatively linked to at least a portion of a PPARA gene, wherein the viral vector upregulates the expression of PPARA to promote lipid metabolism in the hypothalamus of the subject, thereby indirectly altering downstream lipid metabolism in at least a peripheral portion of the subject.

### EXAMPLES

The following are non-limiting examples of the present invention. It is to be understood that said examples are not intended to limit the present invention in any way. Equivalents or substitutes are within the scope of the present invention.

### Example 1: Therapeutic AAV Vector Upregulating Astrocytic PPARα Expression Rescued Hippocampal Synaptic Function in an Amyloidosis Mouse Model of Alzheimer's Disease

Alzheimer's disease mouse model: 5×FAD hemizygous (B6.Cg-Tg (APPSwFILon, PSEN1*M146L*L286V) 6799Vas/Mmjax, Strain #034848-JAX) and its wildtype littermates were produced by crossing with C57BL/6J females.

Virus production: The pAAV-GFAP-mPpara[NM_011144.6]-T2A-mCherry-WPRE was packaged into AAV5 serotype viral vector (FIG. 1 upper construct). The AAV5-GFAP-mPpara[NM_011144.6]-T2A-mCherry-WPRE (1.5×10¹³ GC/mL), and AAV5-GFAP-mCherry-WPRE (1.3×10¹³ GC/mL) were used for injection.

Stereotactic virus injection: 5×FAD hemizygous mice and their wildtype littermates were produced by crossing with C57BL/6J females. 5×FAD mice were injected with AAV particles into bilateral hippocampi at 2 months of age. Briefly, mice were anesthetized with Ketamine (100 mg/kg) and Xylazine (10 mg/kg), and their heads placed in a stereotactic apparatus (KOPF Instruments, USA). The skull was exposed, and a small craniotomy was performed. To cover the hippocampal region, mice were bilaterally microinjected using the following coordinates: anteroposterior (AP), −3.6 mm from bregma, mediolateral (ML), +2.3 mm, dorsoventral (DV), 2.7 mm. All microinjections were carried out using a 5 μL syringe (Hamilton Company, USA) and a glass pipette (WPI, USA). The injection volume and flow rate (100 nL/min) were controlled by an injection pump (WPI). Each mouse received 500 nL of virus on each site individually. Following each injection, the needle was left in place for 10 additional minutes to allow for diffusion of the viral vector away from the needle track and was then slowly withdrawn. The incision was closed using Vetbond tissue adhesive. For postoperative care, mice were subcutaneously injected with Ethiqa XR (3.25 mg/kg).

Immunostaining: Mice brains were perfused and sectioned 5 months after treatment. GFAP antibody was used to identify the distribution and morphological changes of astrocytes. DAPI was used to mark the nuclei.

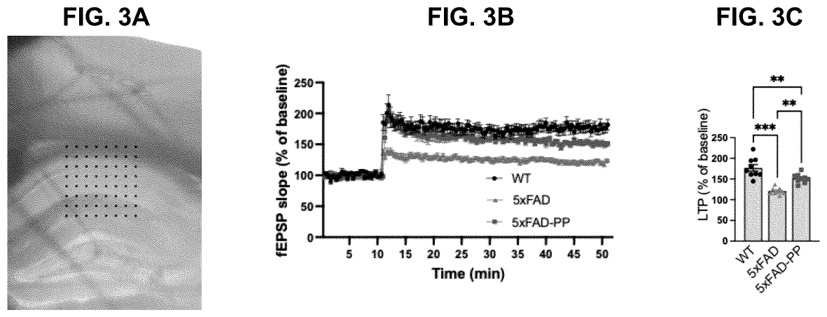

Multi-Electrode Array (MEA) recording: Five months after virus injection mice were anesthetized. Acute hippocampal slices were prepared by a vibratome (Leica VT1200, Leica Microsystems Inc., USA) and positioned over a multi-microelectrode array on a MED64 probe (FIG. 3A) as previously described. Three slices per animal (three animals for each group) were used. Evoked extracellular field recordings were acquired with Med64 Mobius software (Alpha Med Scientific Inc., Japan). Schaffer collateral/commissural pathways were stimulated with biphasic current pulses (200 μs). The Input-Output (I/O) curves were obtained by applying stimuli with increasing amplitudes from 10 to 100 uA. Then stimulation intensity was applied to elicite 30% of the maximum response to evoke fEPSPs. Paired pulse facilitation was measured with two stimulations applied at a different inter-pulse varying from 10 to 50 ms. The percentage of facilitation was determined by calculating the paired-pulse ratio (PPR), i.e., dividing the fEPSP slope of the second response by the fEPSP slope of the first response. Then long-term potentiation (LTP) was induced with a theta burst stimulation (TBS) protocol comprising 10 trains (200 ms duration) of 4 pulses at 5 kHz with 2 s intervals between trains. Before TBS, a stable baseline was established for at least 15 min. The magnitude of LTP was quantified as the percentage change in the fEPSP initial slope (10-40%), measured during the 40-60 min interval after the TBS. Data were analyzed with Mobius software (FIG. 3B).

Statistics: All data were presented as the mean±SEM. Statistical analyses were performed with GraphPad Prism9 (GraphPad Software). Electrophysiology results were compared with two-way ANOVA, followed by Tukey's test for IO curves, and PPRs. LTP analysis was performed by Kruskal-Wallis one-way ANOVA for ranks followed by Dunn's multiple comparisons test. In other experiments, among groups differences were determined with one-way ANOVA, followed by Tukey's multiple comparisons test. P-values<0.05 were considered statistically significant.

The AAV5-GFAP-mPpara[NM_011144.6]-T2A-mCherry-WPRE transduction allows the specific expression of PPARα in astrocytes (FIG. 2). 5×FAD mice were injected with either AAV5-GFAP-mPpara[NM_011144.6]-T2A-mCherry-WPRE virus (5×FAD-PPARα) or vehicle control viruses without the mPpara sequence (5×FAD). Wildtype mice were also injected with control viruses (WT).

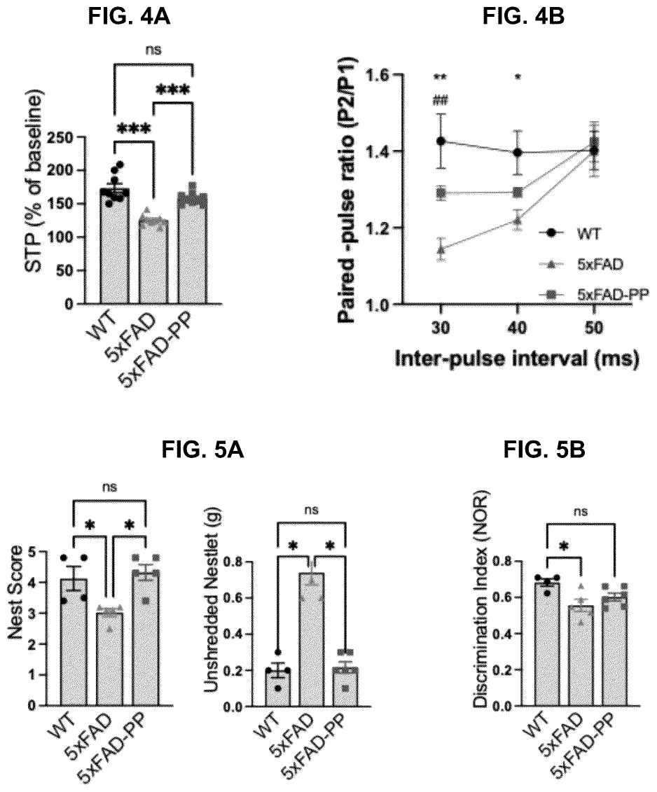

Results from the multielectrode array (MED64) showed that impaired long-term synaptic plasticity and short-term synaptic plasticity in 5×FAD mice are rescued in 5×FAD-PPARα mice (FIG. 3D and FIG. 4A-4B). Field excitatory postsynaptic potentials (fEPSPs) were evoked by stimulating Schaffer collateral fibers and recorded in the CA1 stratum radiatum in slices obtained from 7 months old WT, 5×FAD, and 5×FAD-PPARα mice.

Time course for Theta-burst induced LTP showed that the level of potentiation is dramatically reduced in slices from 7 months old 5×FAD mice compared to slices from WT mice, but partly rescued in 5×FAD-PPARα mice. Kruskal-Wallis one-way ANOVA by ranks followed by Dunn's multiple comparison test (F=41.16) (n=9 slices from 3 animals per group, ***p<0.001, FIG. 3B). Mean LTP was measured from 35 min to 40 min after Theta-burst. The slices from both 5×FAD and 5×FAD-PPARα mice exhibited a reduction in LTP than those from WT mice. The 5×FAD-PPARα group showed a significantly improved LTP response compared to the slices from the 5×FAD group (FIG. 3C). Thus, the upregulation of PPARα in astrocytes rescued the LTP decline in 7 months old 5×FAD mice, which contributes to the improvement in long-term plasticity.

Mean short-term plasticity (STP), measured during 15 min to 20 min post Theta-burst, indicated a significant deficit in 5×FAD mice, which was rescued in 5×FAD-PPARα mice (FIG. 4A). Also, the paired-pulse ratio (PPR) measured at different inter-stimulus intervals (30, 40, and 50 ms) were notably attenuated in slices from 5×FAD mice from 30 and 40 ms intervals stimulation, but not 50 ms stimulation relative to WT controls (FIG. 4B). The PPR differences between 5×FAD-PPARα and WT mice were diminished when the intervals reached more than 30 ms. These suggested that the enhancing PPARα expression in astrocytes improved the impairment of short-term potentiation, as well as PPR in 5×FAD mice, which are important parameters of short-term plasticity.

These data suggest that the enhancing PPARα expression in astrocytes of the hippocampal region improves the long-term and short-term plasticity in Alzheimer's disease mouse models.

### Example 2: Therapeutic AAV Vector Upregulating Astrocytic PPARα Expression Alleviated the Behavioral Impairments in an Amyloidosis Mouse Model of Alzheimer's Disease

Cognitive function: Mice were assessed for novel object recognition (NOR) and novel object placement, tasks that measure recognition memory, presumed to critically involve perirhinal cortex and dorsal hippocampus. Nest-building indexes, which have been identified as highly correlated with motor and hippocampal impairments, were assessed.

Nest building behavior was significantly impaired in 5×FAD mice compared to WT mice, indicative of motor and/or hippocampal impairments (FIG. 5A). Also, NOR test (24 hours interval) indicated a decrease in long-term memory in 5×FAD mice (FIG. 5B). Further, a complete reversal of impairment in nest building and a trend toward improved novel object recognition were observed in 5×FAD-PPARα mice compared to 5×FAD mice (FIG. 5A-5B).

### Example 3: Enhancing PPARα Expression in Hippocampal Astrocytes Reduced Lipid Accumulation and Lipid Peroxidation in an Amyloidosis Mouse Model of Alzheimer's Disease

Immunostaining: The procedure was performed as described in Example 1. PLIN2 antibody was used to indicate the expression and distribution of PLIN2, a lipid droplet marker protein. The PLIN2 expression level has been found highly correlated with triacylglycerol content and lipid droplet load. 4-HNE antibody was applied to determine the levels of 4-HNE, which is a product of lipid peroxidation and is widely accepted as a stable marker for oxidative stress.

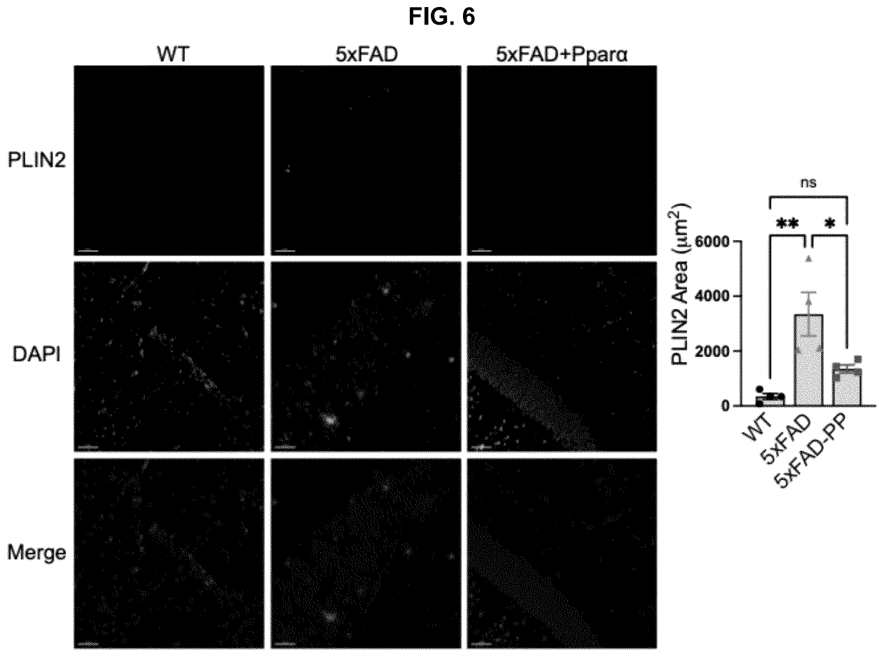

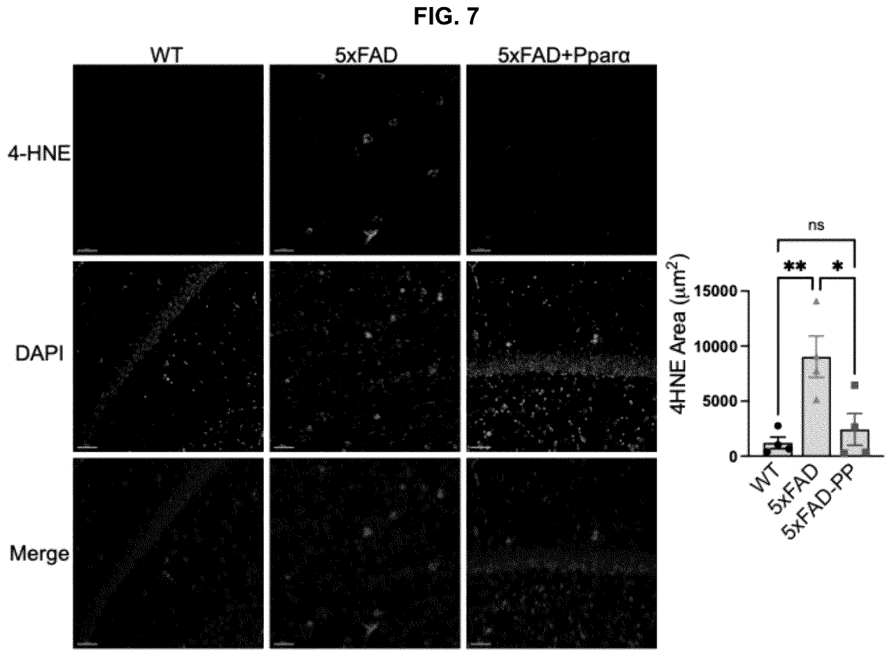

PPARα overexpression in hippocampal astrocytes substantially reduced LD marker PLIN2 (−60%) (FIG. 6) and lipid peroxidation marker 4-HNE (−73%) in the hippocampus (FIG. 7). Thus, by enhancing PPARα expression in hippocampal astrocytes, a remarkable reduction in lipid droplet load and lipid peroxidation was observed in the 5×FAD mouse brains.

### Example 4: Enhancing PPARα Expression in Hippocampal Astrocytes Reduced Amyloid Deposition in an Amyloidosis Mouse Model of Alzheimer's Disease

Immunostaining: The procedure was performed as described in Example 1. Amyloid antibody (6E10) was used to indicate the amount and distribution of amyloid deposition.

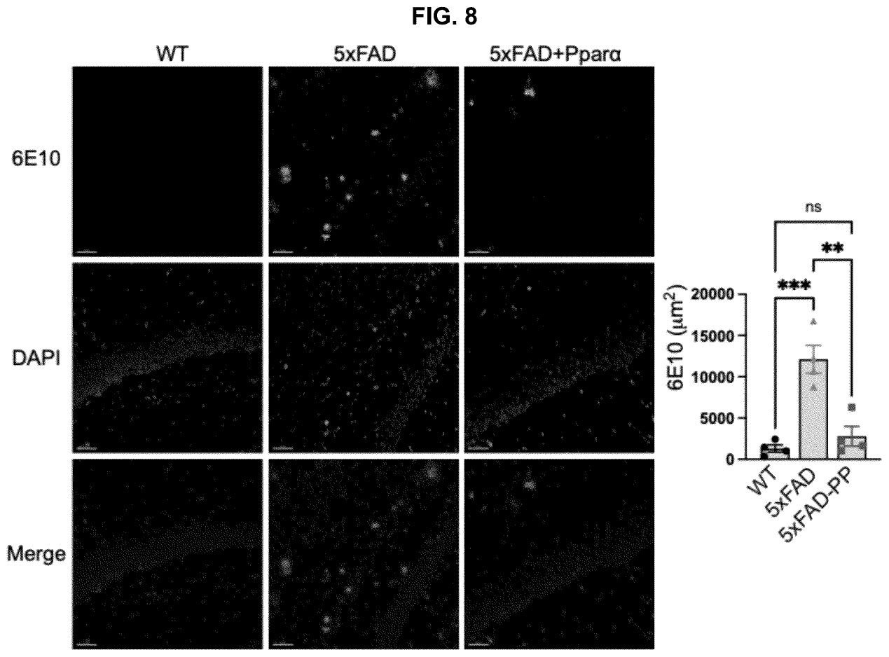

The upregulation of PPARα in hippocampal astrocytes led to a 77% decline in amyloid deposition in the 5×FAD mouse brain (FIG. 8). Selective enhancement of PPARα expression in hippocampal astrocytes effectively reduced amyloid deposition, supporting its potency in mitigating amyloid proteinopathy, a key pathological hallmark of Alzheimer's disease.

Together, these data strongly support that astrocytic PPARα, potentially via increasing FA metabolism and lipid turnover, alleviates AD pathologies and cognitive impairment.

### Example 5: Enhancing PPARα Expression in Hippocampal Astrocytes LED to Prolongation of Both Primary and Secondary Branch Length of Astrocytes in an Amyloidosis Mouse Model of Alzheimer's Disease

Immunostaining: The process is described as Example 1. GFAP antibody was used to indicate the activation, distribution and morphological changes of astrocytes, and the images were analyzed by ImageJ.

Upregulation of PPARα expression in hippocampal astrocytes led to a remarkable rescue of both primary (WT: 22.3±0.707 nm, 5×FAD: 17.9±0.925 nm, 5×FAD-PPARα: 24.9±1.540 nm) and secondary branch lengths (WT: 10.10±0.763 nm, 5×FAD: 6.95±0.534 nm, 5×FAD-PPARα: 11.20±1.040 nm) in 5×FAD mouse brains. Prominent astrocyte reactivity was also observed in 5×FAD mouse brains, which is a common neuropathological finding in Alzheimer's disease, but not in 5×FAD-PPARα mouse brains.

This notable effect suggests the potential of PPARα modulation as a promising therapeutic approach to counteract the adverse effects of Alzheimer's disease-associated hippocampal astrocytes reactivity.

### EMBODIMENTS

The following are non-limiting embodiments of the present invention. It is to be understood that said embodiments are not intended to limit the present invention in any way. Equivalents or substitutes are within the scope of the present invention.

Embodiment 1: A method of treating a neurodegenerative disease in a subject in need thereof, the method comprising administering a viral vector comprising an astrocyte-specific promoter operatively linked to at least a portion of a PPARA gene to a subject diagnosed with a neurodegenerative disease, wherein the viral vector upregulates the expression of PPARA to promote lipid metabolism in the brain.

Embodiment 2: A method of delaying the onset of or preventing a neurodegenerative disease in a subject in need thereof, the method comprising administering a viral vector comprising an astrocyte-specific promoter operatively linked to at least a portion PPARA gene to a subject at risk of having a neurodegenerative disease, wherein the viral vector upregulates the expression of PPARA to promote lipid metabolism in the brain.

Embodiment 3: The method of embodiment 1 or embodiment 2, wherein the neurodegenerative disease is selected from a group consisting of Alzheimer's disease (AD), Parkinson's disease (PD), Huntington's disease (HD), amyotrophic lateral sclerosis (ALS), multiple sclerosis (MS), frontotemporal dementia, Lewy body dementia, Gaucher's disease, progressive supranuclear palsy, stroke, depression, vascular dementia, and prion diseases.

Embodiment 4: The method of any one of embodiments 1-3, wherein the viral vector is an adeno-associated viral (AAV) vector or a lentiviral vector.

Embodiment 5: The method of embodiment 4, wherein the lentiviral vector is selected from a group consisting of AAV2, AAV5, AAV6, AAV9, AAV-PHP.eB, AAV-PHP.B, or AAVrh10 serotypes.

Embodiment 6: The method of any one of embodiments 1-5, wherein the viral vector comprises an astrocyte-specific promoter operatively linked to a full length PPARA gene.

Embodiment 7: The method of any one of embodiments 1-5, wherein the viral vector comprises an astrocyte-specific promoter operatively linked to a fragment of a PPARA gene.

Embodiment 8: The method of any one of embodiments 1-7, wherein the astrocyte-specific promoter is a Glial Fibrillary Acidic Protein (GFAP) promoter.

Embodiment 9: The method of any one of embodiments 1-7, wherein the viral vector is administered intravenously, intranasally, intrathecally, intracisternally, intracerebroventricularly, or a combination thereof.

Embodiment 10: The method of any one of embodiments 1-7, wherein the viral vector is administered via direct injection into the brain.

Embodiment 11: The method of any one of embodiments 1-10, wherein the viral vector is administered via a nanoparticle delivery system.

Embodiment 12: The method of any one of embodiments 1-11, wherein the viral vector is transported across the blood-brain barrier.

Embodiment 13: The method of any one of embodiments 1-12, wherein the viral vector decreases the level of reactive oxygen species (ROS) and oxidative stresses in brain tissues affected by the neurodegenerative diseases.

Embodiment 14: The method of any one of embodiments 1-13, wherein the viral vector diminishes the accumulation of neutral lipid species in brain tissues affected by the neurodegenerative disease.

Embodiment 15: The method of any one of embodiments 1-14, wherein the viral vector increases synaptic function, wherein synaptic function includes short-term and long-term plasticity.

Embodiment 16: The method of any one of embodiments 1-15, wherein the viral vector rescues cognitive function; wherein cognitive function includes cognitive memory and spatial memory.

Embodiment 17: A viral vector comprising an astrocyte-specific promoter operatively linked to at least a portion of a PPARA gene for use in a method of treating a neurodegenerative disease in a subject diagnosed with a neurodegenerative disease, wherein the viral vector upregulates the expression of PPARA to promote lipid metabolism in the brain.

Embodiment 18: A viral vector comprising an astrocyte-specific promoter operatively linked to at least a portion of a PPARA gene for use in a method of preventing a neurodegenerative disease in a subject diagnosed with a neurodegenerative disease, wherein the viral vector upregulates the expression of PPARA to promote lipid metabolism in the brain.

Embodiment 19: The viral vector of embodiment 17 or embodiment 18, wherein the neurodegenerative disease is selected from a group consisting of Alzheimer's disease (AD), Parkinson's disease (PD), Huntington's disease (HD), amyotrophic lateral sclerosis (ALS), multiple sclerosis (MS), frontotemporal dementia, Lewy Body dementia, Gaucher's disease, progressive supranuclear palsy, stroke, depression, vascular dementia, and prion diseases.

Embodiment 20: The viral vector of any one of embodiments 17-19, wherein the viral vector is an adeno-associated viral (AAV) vector or a lentiviral vector.

Embodiment 21: The method of embodiment 4, wherein the lentiviral vector is selected from a group consisting of AAV2, AAV5, AAV6, AAV9, AAV-PHP.eB, AAV-PHP.B, or AAVrh10 serotypes.

Embodiment 22: The viral vector of any one of embodiments 17-21, wherein the viral vector comprises an astrocyte-specific promoter operatively linked to a full length PPARA gene.

Embodiment 23: The viral vector of any one of embodiments 17-21, wherein the viral vector comprises an astrocyte-specific promoter operatively linked to a fragment of a PPARA gene.

Embodiment 24: The viral vector of any one of embodiments 17-23, wherein the astrocyte-specific promoter is a Glial Fibrillary Acidic Protein (GFAP) promoter.

Embodiment 25: The viral vector of any one of embodiments 17-24, wherein the viral vector is administered intravenously, intranasally, intrathecally, intracisternally, intracerebroventricularly, or a combination thereof.

Embodiment 26: The viral vector of any one of embodiments 17-24, wherein the viral vector is administered via direct injection into the brain.

Embodiment 27: The viral vector of any one of embodiments 17-24, wherein the viral vector is administered via a nanoparticle delivery system.

Embodiment 28: The viral vector of any one of embodiments 17-27, wherein the viral vector is transported across the blood-brain barrier.

Embodiment 29: The viral vector of any one of embodiments 17-28, wherein the viral vector decreases the level of reactive oxygen species (ROS) and oxidative stresses in brain tissues affected by the neurodegenerative diseases.

Embodiment 30: The viral vector of any one of embodiments 17-29, wherein the viral vector diminishes the accumulation of neutral lipid species in brain tissues affected by the neurodegenerative disease.

Embodiment 31: The viral vector of any one of embodiments 17-30, wherein the viral vector increases synaptic function, wherein synaptic function includes short-term and long-term plasticity.

Embodiment 32: The viral vector of any one of embodiments 17-31, wherein the viral vector rescues cognitive function; wherein cognitive function includes cognitive memory and spatial memory.

Embodiment 33: Use of a viral vector comprising an astrocyte-specific promoter operatively linked to at least a portion of a PPARA gene in the manufacture of a medicament for the treatment of a neurodegenerative disease.

Embodiment 34: The viral vector of embodiment 33, wherein the neurodegenerative disease is selected from a group consisting of Alzheimer's disease (AD), Parkinson's disease (PD), Huntington's disease (HD), amyotrophic lateral sclerosis (ALS), multiple sclerosis (MS), frontotemporal dementia, Lewy Body dementia, Gaucher's disease, progressive supranuclear palsy, stroke, depression, vascular dementia, and prion diseases.

Embodiment 35: The viral vector of embodiment 33 or embodiment 34, wherein the viral vector is an adeno-associated viral (AAV) vector or a lentiviral vector.

Embodiment 36: The method of embodiment 35, wherein the lentiviral vector is selected from a group consisting of AAV2, AAV5, AAV6, AAV9, AAV-PHP.eB, AAV-PHP.B, or AAVrh10 serotypes.

Embodiment 37: The viral vector of any one of embodiments 33-36, wherein the viral vector comprises an astrocyte-specific promoter operatively linked to a full length PPARA gene.

Embodiment 38: The viral vector of any one of embodiments 33-36, wherein the viral vector comprises an astrocyte-specific promoter operatively linked to a fragment of a PPARA gene.

Embodiment 39: The viral vector of any one of embodiments 33-38, wherein the astrocyte-specific promoter is a Glial Fibrillary Acidic Protein (GFAP) promoter.

Embodiment 40: The viral vector of any one of embodiments 33-39, wherein the viral vector is administered intravenously, intranasally, intrathecally, intracisternally, intracerebroventricularly, or a combination thereof.

Embodiment 41: The viral vector of any one of embodiments 33-40, wherein the viral vector is administered via direct injection into the brain.

Embodiment 42: The viral vector of any one of embodiments 33-40, wherein the viral vector is administered via a nanoparticle delivery system.

Embodiment 43: The viral vector of any one of embodiments 33-40, wherein the viral vector is transported across the blood-brain barrier.

Embodiment 44: The viral vector of any one of embodiments 33-43, wherein the viral vector decreases the level of reactive oxygen species (ROS) and oxidative stresses in brain tissues affected by the neurodegenerative diseases.

Embodiment 45: The viral vector of any one of embodiments 33-44, wherein the viral vector diminishes the accumulation of neutral lipid species in brain tissues affected by the neurodegenerative disease.

Embodiment 46: The viral vector of any one of embodiments 33-45, wherein the viral vector increases synaptic function, wherein synaptic function includes short-term and long-term plasticity.

Embodiment 47: The viral vector of any one of embodiments 33-46, wherein the viral vector rescues cognitive function; wherein cognitive function includes cognitive memory and spatial memory.

Embodiment 48: A method of screening for a PPARα agonist, the method comprising: obtaining a cell line comprising a PPARα-responsive promoter operatively linked to a reporter gene; and contacting the cell line with a potential PPARα agonist compound; wherein a potential PPARα agonist compound is confirmed as a PPARα agonist when expression of the reporter gene increases compared to a vehicle-treated control.

Embodiment 49: The method of embodiment 48, wherein the cell line is a HepG2.

Embodiment 50: The method of embodiment 48, wherein the reporter gene is a luciferase.

Embodiment 51: A method of delaying the onset of, treating, or preventing a neurodegenerative disease in a subject in need thereof, comprising administering to the subject a therapeutically effective amount of a viral vector comprising a nucleic acid molecule encoding for one or more homologous genes encoding PPARα which upregulate the expression and/or activity of PPARα selectively inside of astrocytes in the central nervous system.

Embodiment 52: The method of embodiment 51, wherein the viral vector further comprises an astrocytes specific promoter operatively linked to the PPARα nucleic acid molecule.

Embodiment 53: The method of embodiment 52, wherein the astrocyte-specific promoter is a Glial Fibrillary Acidic Protein (GFAP) promoter.

Embodiment 54: The method of any one of embodiment 51-53, wherein nucleic acid molecule encoding for one or more homologous genes upregulates expression of PPARα and its functional fragments or isoforms.

Embodiment 55: The method of any one of embodiment 51-54, wherein the viral vector is an adeno-associated viral (AAV) vector or a lentiviral vector.

Embodiment 56: The method of embodiment 55, wherein the lentiviral vector is selected from a group consisting of AAV2, AAV5, AAV6, AAV9, AAV-PHP.eB, AAV-PHP.B, or AAVrh10 serotypes.

Embodiment 57: The method of any on of embodiment 51-56, wherein the neurodegenerative disease is selected from a group consisting of Alzheimer's disease (AD), Parkinson's disease (PD), Huntington's disease (HD), amyotrophic lateral sclerosis (ALS), multiple sclerosis (MS), frontotemporal dementia, Lewy Body dementia, Gaucher's disease, progressive supranuclear palsy, stroke, depression, vascular dementia, and prion diseases.

Embodiment 58: The method of any one of embodiment 51-57, wherein the viral vector is administered intravenously, intranasally, intrathecally, intracisternally, intracerebroventricularly, or a combination thereof.

Embodiment 59: The method of any one of embodiment 51-57, wherein the viral vector is administered via direct injection into the brain.

Embodiment 60: The method of any one of embodiment 51-57, wherein the viral vector is administered via a nanoparticle delivery system.

Embodiment 61: The method of any one of embodiment 51-57, wherein the viral vector is transported across the blood-brain barrier.

Embodiment 62: The method of any one of embodiments 51-61, wherein the viral vector is transported across the blood-brain barrier.

Embodiment 63: The method of any one of embodiments 51-62, wherein the viral vector decreases the level of reactive oxygen species (ROS) and oxidative stresses in brain tissues affected by the neurodegenerative diseases.

Embodiment 64: The method of any one of embodiments 51-63, wherein the viral vector diminishes the accumulation of neutral lipid species in brain tissues affected by the neurodegenerative disease.

Embodiment 65: The method of any one of embodiments 51-64, wherein the viral vector increases synaptic function, wherein synaptic function includes short-term and long-term plasticity in the subject.

Embodiment 66: The method of any one of embodiments 51-65, wherein the viral vector rescues cognitive function; wherein cognitive function includes cognitive memory and spatial memory.

Embodiment 67: The method of embodiment 1, wherein the viral vector comprises the polynucleotide of SEQ ID NO: 1.

Embodiment 68: A viral vector comprising an astrocyte-specific promoter operatively linked to at least a portion of a PPARA gene for use in a method of treating or delaying the onset of or preventing a neurodegenerative disease in a subject diagnosed with a neurodegenerative disease, wherein the viral vector upregulates the expression of PPARA to promote lipid metabolism selectively inside of astrocytes in the central nervous system.

Embodiment 69: The viral vector of embodiment 68, wherein the viral vector comprises the polynucleotide of SEQ ID NO: 1.

Embodiment 69: A method of treating peripheral metabolic dysregulation associated with neurodegenerative disease in a subject in need thereof, the method comprising administering a viral vector comprising an astrocyte-specific promoter operatively linked to at least a portion of a PPARA gene to a hypothalamus of a subject diagnosed with a neurodegenerative disease, wherein the viral vector upregulates the expression of PPARA to promote lipid metabolism in the hypothalamus of the subject, thereby indirectly altering downstream lipid metabolism in at least a peripheral portion of the subject.

Embodiment 70: The method of embodiment 69, wherein the viral vector is an adeno-associated viral (AAV) vector or a lentiviral vector.

Embodiment 71: The method of embodiment 69, wherein the viral vector comprises the polynucleotide of SEQ ID NO: 1.

Embodiment 72: The method of embodiment 69, wherein the viral vector comprises an astrocyte-specific promoter operatively linked to at least a fragment of a PPARA gene.

Embodiment 73: The method of embodiment 69, wherein the viral vector comprises a nucleotide sequence with at least 90% sequence identity to SEQ ID NO: 1, SEQ ID NO: 3, or SEQ ID NO: 7.

Embodiment 74: The method of embodiment 69, wherein the viral vector comprises a nucleotide sequence with at least 90% sequence identity to a segment of SEQ ID NO: 1 beginning at nucleotide 869 and ending at nucleotide 2272.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of treating or delaying the onset of or preventing a neurodegenerative disease in a subject in need thereof, the method comprising administering a viral vector comprising an astrocyte-specific promoter operatively linked to at least a portion of a PPARA gene to the subject, wherein the viral vector upregulates the expression of PPARα to promote lipid metabolism selectively inside of astrocytes in the central nervous system.

2. The method of claim 1, wherein the neurodegenerative disease is selected from a group consisting of Alzheimer's disease (AD), Parkinson's disease (PD), Huntington's disease (HD), amyotrophic lateral sclerosis (ALS), multiple sclerosis (MS), frontotemporal dementia, Lewy body dementia, Gaucher's disease, progressive supranuclear palsy, stroke, depression, vascular dementia, and prion diseases.

3. The method of claim 1, wherein the viral vector is an adeno-associated viral (AAV) vector or a lentiviral vector.

4. The method of claim 3, wherein the AAV vector is selected from a group consisting of AAV2, AAV5, AAV6, AAV9, AAV-PHP.eB, AAV-PHP.B, or AAVrh10 serotypes.

5. The method of claim 1, wherein the viral vector comprises the astrocyte-specific promoter operatively linked to at least a functional fragment of a PPARA gene.

6. The method of claim 5, wherein the astrocyte-specific promoter is a Glial Fibrillary Acidic Protein (GFAP) promoter.

7. The method of claim 1, wherein the viral vector is administered intravenously, intranasally, intrathecally, intracisternally, intracerebroventricularly, or a combination thereof.

8. The method of claim 1, wherein the viral vector is administered via direct injection into the subject's brain.

9. The method of claim 1, wherein the viral vector is administered via a nanoparticle delivery system.

10. The method of claim 9, wherein the nanoparticle delivery system is configured to transport the viral vector across the blood-brain barrier.

11. The method of claim 1, wherein the viral vector decreases the level of reactive oxygen species (ROS) and oxidative stresses in brain tissues affected by the neurodegenerative diseases.

12. The method of claim 1, wherein the viral vector diminishes the accumulation of neutral lipid species in brain tissues affected by the neurodegenerative disease.

13. The method of claim 1, wherein the viral vector increases synaptic function, wherein synaptic function includes short-term and long-term plasticity.

14. The method of claim 1, wherein the viral vector rescues cognitive function; wherein cognitive function includes cognitive memory and spatial memory.

15. The method of claim 1, wherein the viral vector comprises a nucleotide sequence with at least 90% sequence identity to SEQ ID NO: 1, SEQ ID NO: 3, or SEQ ID NO: 7.

16. The method of claim 1, wherein the viral vector comprises a nucleotide sequence with at least 90% sequence identity to a segment of SEQ ID NO: 1 beginning at nucleotide 869 and ending at nucleotide 2272.

17. A method of treating peripheral al metabolic dysregulation associated with neurodegenerative disease in a subject in need thereof, the method comprising administering a viral vector comprising an astrocyte-specific promoter operatively linked to at least a portion of a PPARA gene to a hypothalamus of a subject diagnosed with a neurodegenerative disease, wherein the viral vector upregulates the expression of PPARα to promote lipid metabolism in the hypothalamus of the subject, thereby indirectly altering downstream lipid metabolism in at least a peripheral portion of the subject.

18. The method of claim 17, wherein the viral vector comprises the astrocyte-specific promoter operatively linked to at least a functional fragment of a PPARA gene.

19. The method of claim 17, wherein the viral vector comprises a nucleotide sequence with at least 90% sequence identity to SEQ ID NO: 1, SEQ ID NO: 3, or SEQ ID NO: 7.

20. The method of claim 17, wherein the viral vector comprises a nucleotide sequence with at least 90% sequence identity to a segment of SEQ ID NO: 1 beginning at nucleotide 869 and ending at nucleotide 2272.


SEQUENCE LISTING (7 sequences)

SEQ ID NO: 1 - DNA, 6668 bp, synthetic construct
  source 1..6668: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccaagtttgtacaaaaaagcaggctgccaccatggtggacacagagagccccatctgtcctctctccccactggaggcagatgacctggaaagtcccttatctgaagaattcttacaagaaatgggaaacattcaagagatttctcagtccatcggtgaggagagctctggaagctttggttttgcagactaccagtacttaggaagctgtccgggctccgagggctctgtcatcacagacaccctctctccagcttccagcccttcctcagtcagctgccccgtgatccccgccagcacggacgagtcccccggcagtgccctgaacatcgagtgtcgaatatgtggggacaaggcctcagggtaccactacggagttcacgcatgtgaaggctgtaagggcttctttcggcgaactattcggctgaagctggtgtacgacaagtgtgatcggagctgcaagattcagaagaagaaccggaacaaatgccagtactgccgttttcacaagtgcctgtctgtcgggatgtcacacaatgcaattcgctttggaagaatgccaagatctgaaaaagcaaaactgaaagcagaaattcttacctgtgaacacgacctgaaagattcggaaactgcagacctcaaatctctgggcaagagaatccacgaagcctacctgaagaacttcaacatgaacaaggtcaaggcccgggtcatactcgcgggaaagaccagcaacaacccgccttttgtcatacatgacatggagaccttgtgtatggccgagaagacgcttgtggccaagatggtggccaacggcgtcgaagacaaagaggcagaggtccgattcttccactgctgccagtgcatgtccgtggagaccgtcacggagctcacagaatttgccaaggctatcccaggctttgcaaacttggacttgaacgaccaagtcaccttgctaaagtacggtgtgtatgaagccatcttcacgatgctgtcctccttgatgaacaaagacgggatgctgatcgcgtacggcaatggctttatcacacgcgagttccttaagaacctgaggaagccgttctgtgacatcatggaacccaagtttgacttcgctatgaagttcaatgccttagaactggatgacagtgacatttccctgtttgtggctgctataatttgctgtggagatcggcctggccttctaaacataggctacattgagaagttgcaggaggggattgtgcacgtgcttaagctccacctgcagagcaaccatccagatgacaccttcctcttcccaaagctccttcaaaaaatggtggaccttcggcagctggtcacggagcatgcgcagctcgtacaggtcatcaagaagaccgagtccgacgcagcgctgcacccactgttgcaagagatctacagagacatgtacggaagcggagagggcaggggaagtcttctaacatgcggggacgtggaggaaaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 2 - DNA, 681 bp, Homo sapiens
  source 1..681: mol_type = genomic DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 3 - DNA, 1404 bp, Mus musculus
  source 1..1404: mol_type = genomic DNA
atggtggacacagagagccccatctgtcctctctccccactggaggcagatgacctggaaagtcccttatctgaagaattcttacaagaaatgggaaacattcaagagatttctcagtccatcggtgaggagagctctggaagctttggttttgcagactaccagtacttaggaagctgtccgggctccgagggctctgtcatcacagacaccctctctccagcttccagcccttcctcagtcagctgccccgtgatccccgccagcacggacgagtcccccggcagtgccctgaacatcgagtgtcgaatatgtggggacaaggcctcagggtaccactacggagttcacgcatgtgaaggctgtaagggcttctttcggcgaactattcggctgaagctggtgtacgacaagtgtgatcggagctgcaagattcagaagaagaaccggaacaaatgccagtactgccgttttcacaagtgcctgtctgtcgggatgtcacacaatgcaattcgctttggaagaatgccaagatctgaaaaagcaaaactgaaagcagaaattcttacctgtgaacacgacctgaaagattcggaaactgcagacctcaaatctctgggcaagagaatccacgaagcctacctgaagaacttcaacatgaacaaggtcaaggcccgggtcatactcgcgggaaagaccagcaacaacccgccttttgtcatacatgacatggagaccttgtgtatggccgagaagacgcttgtggccaagatggtggccaacggcgtcgaagacaaagaggcagaggtccgattcttccactgctgccagtgcatgtccgtggagaccgtcacggagctcacagaatttgccaaggctatcccaggctttgcaaacttggacttgaacgaccaagtcaccttgctaaagtacggtgtgtatgaagccatcttcacgatgctgtcctccttgatgaacaaagacgggatgctgatcgcgtacggcaatggctttatcacacgcgagttccttaagaacctgaggaagccgttctgtgacatcatggaacccaagtttgacttcgctatgaagttcaatgccttagaactggatgacagtgacatttccctgtttgtggctgctataatttgctgtggagatcggcctggccttctaaacataggctacattgagaagttgcaggaggggattgtgcacgtgcttaagctccacctgcagagcaaccatccagatgacaccttcctcttcccaaagctccttcaaaaaatggtggaccttcggcagctggtcacggagcatgcgcagctcgtacaggtcatcaagaagaccgagtccgacgcagcgctgcacccactgttgcaagagatctacagagacatgtac

SEQ ID NO: 4 - DNA, 63 bp, Thosea asigna virus
  source 1..63: mol_type = genomic DNA
ggaagcggagagggcaggggaagtcttctaacatgcggggacgtggaggaaaatcccggcccc

SEQ ID NO: 5 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 6 - DNA, 598 bp, Woodchuck hepatitis virus
  source 1..598: mol_type = genomic DNA
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 7 - DNA, 10118 bp, Homo sapiens
  source 1..10118: mol_type = genomic DNA
gtggacgcggcggccccgcggcgggggcagcgggcggcgggggcggaggcggccgctagcgccctgcccggcgccgcctccttcggcgttcgccccacggaccggcaggcggcggaccgcggcccaggctgaagctcagggccctgtctgctctgtggactcaacagtttgtggcaagacaagctcagaactgagaagctgtcaccacagttctggaggctgggaagttcaagatcaaagtgccagcagattcagtgtcatgtgaggacgtgcttcctgcttcatagataagagtagcttggagctcggcggcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctgggggtaggtggctaatctcaggactgggaagattacggcgaattatgctcaatggtctgattttaactcacccgatgttaatcaatgcacattgctttagatcacattcgtgatttaccatttaattaactggtaacctcaaaattcgtggcctgtcttcccattcaccccgcttttgactattgtgctcctttataattctgaaaactaatcagcactttttaacaatgtttataatcctataagtctagatgtatccaaaggtgaagtatgtaaaaagcagcaaaatatttatttcaaagacttcacttctgtttcctgaatctaaagaaagacaacatgctgctttttaatcataggatggagaattttaaagaactgtttgggccaggcacagtcgctcatacttgtaatcccagcactttgggaggccgaggcgggtggatcacaaggtcagcagatcgagaccatcctggccaacatggtgaaaccctgtctctactaaaaatacaaaaattagccgggtgtggtggcacatgcctgtaatcccagctactcgggaagctgaggcaggagaattgcttgaaccagggagttggaggttgcagtgagctaagactgcaccactgcactccagcctggtgacagaacgagactctgtcttaaaaacaaacaaacaaaaaaaaaatctgttagataagctatcaaaatgcagctgttgttttgtttttggctcactgttttcgtggttgtaactaatatgtggaaaggcccatttccaggtttgcgtagaagagcccagaaaacagagtctcaagacccccgctctggactgtcataagctagcacccgtggtaagcgggacgagacaagctcccgaagcccgccagcttcctgctccactcagctccgtccagtcaacctgaacccacccagtccagctgtctgtgggaatggtggtgttcttagggacagactgacaccttacttgtcagtgttcctccgggccccatttggcagctcccgtatcttttgttatgttgcttttaaagatatgatgttttattgttttaactcttggtgacagtagatgctctctggagcgcagacgaggcacatgtgtcttcatagcctgggctgggtgggagccagtcaccctgcggatcgagagagggggtagagtcttcttcaaatggcagttttacttcaaatggcagatttcacaagagttggttattttttacaatggtttaggttgttaagtctcctttgtatgtaaggtagttttttcaacatctaaaatttttgttttagccttcaaaaccaacttaccaacctcagtccagctgggaaggcagcgttgattatggtagtttgtcaagaatatatggacctggaaacactttctctctctgtccacctggtagataaattgtcctgttgagaatttttagatctggactggaactgccaggaccaccgcctccagggagtcgctgggcacctggaggtatcgtcgatgcctctcccccatctttagaaaatttggctcttctgaggtcattattattttaagaatgattaggattgataagggtcccatgaccagcattatgaaaatgcgagagtgggaaggacacagtgtgagacttccactagaaaaaagtgaaagttagggttaggacatccttttttaaaaattacaaatttagtccgttttggtttttgtaatcaggctaggcacagtggctcacacatggaatcccagcactttgggaggccgaggtgggaggatcacttgagcccaggagttcgagaccagcctaggcaacatagcaagaccctgtctgtacacaaaatttaaaaattagttcatcggggtggcacacatcagtagtcccagctactctgcaggctgaggtgggaggattgcttgaacccaggaggtcgaggctgcagtgagctgtgatctcaccactgcattccagcctgggtgacagagttagattccaccctctcccaccccggcaaaaaaaaaaaaaaaagatgcaatcaaaggggctgttggccagcaatggcagcagcagcggcgggcagtctgcccaagtgtcttaggaaccaaaagcaaataaaagtgtttccatatatgccaccagccaagtggccatcctaattcagaaagaagctagcctttgagtgtctgtcatggtgcatccgtttcagtattatttcctaaaatgagaagcccctgtgtcaacaagatccaggggctggagcccaatgccaagcctgtgttgtccccagcgaccctgcagctgctcgctctgatgtaccctgtgccattcaaggagatgtggtccaggaaagtgagcctcatggttttcagagaagtcattgttctgtttacattttcataaaacctgtttaaaatagctccccgtctcaggctttcagcagtaacagtgagctgactggcaagttcgatgttagctcccgggacactcagcagcgatggtgagcattttggtttccttaaggcccagcaagacttccagggacatctctggtgaagccagaatggagacacccgtgacctcaggctgaaagtcactcgacattggtctcttgtgttgatagggaaggaaatcaggcattcctatttctttaaataacaaaaccactaattgccactcaatgctggaatattttgggtcacctaatcatagatttctcagggcatcaatactcaaatataggctgattatgccccagttcaaatgggaactattaacagagtgcatttcttgcttgctgggtttcaacagacatcagccaaaagaacaaaagagatgtcaggacagattccaggagtgtcggagcacatgtgtggcacccgctccctctggcagcgaatgtaggaagtcgccaaatttacccactcttcaacaagtcattgtttaaacacggtttttcattttctcaacttttaatagcaaaaagtgccaaagtcctcagagacctaacagccttggtctaccgtgctgaccagggtgaaggcacggcgagggactcctcccagacgtgcctcttgtgtgccagctggctgtggctcgggagcagacgcaggcctctccattgtccaggggagcctggcggcgcatccctcctctcccacctcctggcacttccagctgggtgtcccacatgttggattccgtccccaccacacttccagagaccggagaactgtgcagggcctaaggccgtttggatgaattgtcaaaacaagatgcttccagttacagcggcaggagcgggactgggagcacgggctgacggctgctggtgcctttcttcccacctcgcttgcctgtttccgcttgacccttcctccagctccgatgagaagagtataaagcatcttcctaacgggtgtgtttgctatacgaacataatggacgtgaagtggggcagaaacccagaactcagcattcaaggatgcccaggagagctgtccctgttttaaagagctgtgttttgttttgtttcgcatttagagagcagacaaggcacccttctgctgcgctgatacgtttcttacactgggccattttagacccccagggaaacagccttcctggagcgttgtctggaggttccagggacagggcagcctcccagagccgagcaagagctcaaggtacaaatgagagatttgctataccgtgagaagtcaacaacttagccaccacttccccgcaatggaccatgtaacaaatacctcagcaggccctgcaaaaggccatgctagagctgaggcgcacagcctgtggcctctgtagttagggcaggtgggatggagactccttgagtgcacacacctgagcctgcccacacacaggggagcagcatctcgtatgacgtctggaaggaacttcggttgtgtaaagggagccttgaagatacgtgcaaaaggtgctaccccaatttggtgaaactgacattgggcacgtcttgggcttaggagaagcggccgatggtcccggcctgcagtgacaaacccccctccccgcaccgcccccagcaccccctctcctcttcacctcttcctgctggccacgaggaagccacttcctcagagagaccctaccagatgcggatggaaacagatgcaccaaagcaagccctgatgaaaccgcgacttcctaaggtctgtctcctctgaacttgcacctgggcctctctgtgtttggttccaagcacttcccacctcaaactcccattttcaaaccactgtatctctgcgcacatctgctacttaccagccgcatacatgatggagggttttttggtcctgatccagtggccacacctgtctttgaaatgtctcactgaactccagttttaaaatagattcattgcttcaacacagcaagcccaatgcacccagctaagactggcttgaccgacagcctggcctttggtggggggcttcctggggcctggggaaagctggccaccttcaacagctggtacctcttcaacagtgtggcctttcaaaatgcagatgccaccaggagaacatgcccacagctcaccacctatggatgccatggctctgggcagctttcaaagcaggttcctgtggtctcctcagctgtttgagggggtaacagcaaatcagcctccattttaaaatgaaaacaccagcctccagatgtagggcctgctgggtgttgctagccgctggtccccaggcacggtgcactttctccacctcctgcagcctccctgttgtttctagactcttgcacctggtgagtgcaaggataggtgacccaggggcctgcagccttgtcctcagctcccatctcctggactgccagcctcaccctctgcagttagcatggttggcctgatgcagggatcccgagggattactttttagaccttctttcacattcagaaaagtagtatagattcaggagaggcaagaaaattatgctgtccatagaagtcacccatgaagactgatgccaccacctgaaggctcatgattgttaaaaatgtccacgggaacctctcgtccacaggaggtttgtctcaacacttcccatttttacggcattggcattgccaagcatggggaagtatctgctcttctcatgttaaaagtggcccagcttttcttaactcagtccaagctgacttgtttagctgcactggaatttcttaccaaccaaatatttgcatcgagcaaagggggctgtgtgcacctccctaatggcagcgatgatggctgctgtcattcaagcccatcttcagacgtcacagtctggaagtgaaatgtccacaaacatctgtggcagaaaaggctatacggaccacccagttgtgctgcagctttacagagcaaggaagggttgtggcaaataaatgattaacctgcctcgactgtgctgagggcaacaaaggccatctcaccaaaggattattcgatgccattaaatcatcccgtgaccttcctgcttccgagtccatggcctttgcccagggcatgtactcccctgagaggccttctgcctagaaagatctatgactgggttccaaagttgaggcctaggtttttgctgggatttagatattttcaggcaccattttgacagcattcaggaaaacggttattgaccccatagactagggtaagaataaaggcaataaatttggtctgactcagaatataggagatccatatatttctctggaaaccacagtgtacactaaaatgtgaaattgaaggttttgttaaaaagaaaaagataatgagcttcatgctttgtttaattacataatgatttccattacgctatttctgtgaaatgcagcaggttcttaaacgttatttcagtggcatgggctggaagcttatcacaaaaagccatgtgtgtggccttatcagaacagaaagagacaggctggtgcccaaggctgctgcctgctccaccttttgccagctctggacatctgaggacgtcccggcagatctggaatggggccctcaactgaccatttgcttctcagaatttcagtttgagacatgagaggtataatcagttacttttctccccccagagaaacccttttgtgaggggagaggagctatggtatgtggttcagctgaaacacatacaactgcatccttttggagtcctttgccaacaaaaacagaccaacagaccagatggtgtccatgttcaatatcatgtcttgatggacgcagctgatgacctcaaatacttgagtggtctcatggctgttagatggattatttgaaaaaaaaaaaaaaaaaagagagaaaaaataattgatttttacatcagagatagcaaactaagacctggggaggggggtcagcttttattttattttattttttttaagtttgctagttgggtcaaatgtgaggaggagggagtctacctgccacctcttctcttgcccctcttctgcccacacatccagcatccaaaatccattcatttaatgaattgataaagtgccgtgcaaactggtgcacaaacaggcccccagtccacgcagcctggctcctaggaaaagtggtgaccgggcgtgggggggcatgccgcagccctgggacacagtcgggcaccttccccggacccccaggccttggctgtgcctcaagtcagagagggtcagccttcaggccccggagacgagtgactggccgatcatttcacaataaaatcactcacttttggcaacttcactttttttaaggcacagtcagttccttttctcatgtacctcacaaaagatgaagaccatgtagtactctttttggtaaagttacagtgttcatgttaaatatcacttttttctacattgtgtggtaaaaagaactacgttaatagctatatcttaaatactgtgatttgactttttgaaaaatatcctaatacaaatattttactaacttacaatcactcatttaataagaaacatttggattcttttgaaatcagtgttaattgactcatattcttaaaagcctggctcttgaccctattggaaacacaaaggaagctgaaatcaaacatctaaaatacactgcgtacacgtgtgcgtgcacacacacacacacacacacacacacacagctcttcatttctcctgagccatgcagaatttactttcaatgtggaaatctgttccctttaccacactgtatatgcacagagcacaagagaggctatctctagtcacttccaccagcgaggccttagactccgtattagaggccaccgatttcatacaacagtgtttcgctaaagacccttcactattcttgtttagtaaatagctgtctgctcttcagggaactgttacctatgggttattaccaaagaacgctggcaattggaaatgtcctgatggaaattctttgcacgtgccggttctctggcatcctccaggtggcccaacccaaagcagaaagcagaaaccacagaccccgtgagtctccccataccttgtttccaataacttggcaaaacttcttggtgcatattggttacaccctctgggattcataatgccattaggctaaaaccctaagagagagggttgacagaaacacacgcgagaatgaggcagatcccagagcaaggactgggcccagactctccacatgtgctctactagtgagtgccttatactctcagtattttggggcttacagcttcttatttgtgctaaaaaggtgcagttccaaagtaggaactgccacacaggccccagcatcctctctccaacttcatacctctctcctggtggggggagcgggcatccaggacctccggaatcaaggatgtgcagagaagagcgaaagtaatttttctagtcacatgaactgattggttccaggcaattagaaaatggctataaaataaccttaattttaaaaaaaaatcttgggtcttcgttttcctattaggagactgaactgaccacatgtattgatttatatcctgaatatatgggaacttctgtgtttgggatgtcctactgtaagactgatgaatgtacagagttaatttcagggtacagttttgccttaatggttttaaaaaataaactattttttaaaattt